(19) United States
(12) Patent Application Publication
(10) Pub. No.: US 2019/0167179 A1
(43) Pub. Date: Jun. 6, 2019

(54) METHODS AND SYSTEM FOR ASSESSING A COGNITIVE FUNCTION

(71) Applicant: Hadasit Medical Research Services and Development Ltd., Jerusalem (IL)

(72) Inventor: Shahar ARZY, Jerusalem (IL)

(73) Assignee: Hadasit Medical Research Services and Development Ltd., Jerusalem (IL)

(21) Appl. No.: 16/323,791

(22) PCT Filed: Aug. 7, 2017
(86) PCT No.: PCT/IL2017/050872

§ 371 Date: Feb. 7, 2019

Related U.S. Application Data
(60) Provisional application No. 62/371,784, filed on Aug. 7, 2016.

(51) Int. Cl. A61B 5/00

(52) U.S. Cl. CPC A61B 5/4088; A61B 5/4064; A61B 5/7267; A61B 5/16; A61B 5/742; A61B 5/0022; A61B 5/4848; A61B 5/7475

(57) ABSTRACT

A method of neuropsychological analysis comprises: presenting to a subject, by a user interface, a subject-specific cognitive task having at least one task portion selected from the group consisting of a time-domain task portion, a space-domain task portion, and a person-domain task portion. The method also comprises receiving responses entered by the subject using the user interface for each of the task portions, representing the responses as a set of parameters, and classifying the subject into one of a plurality of cognitive function classification groups, based on the set of parameters.

### RELATED APPLICATION

This application claims the benefit of priority of U.S. Provisional Patent Application No. 62/371,784 filed Aug. 7, 2016, the contents of which are incorporated herein by reference in their entirety

### FIELD AND BACKGROUND OF THE INVENTION

The present invention, in some embodiments thereof, relates to neuromedicine and, more particularly, but not exclusively, to a method and system for assessing a cognitive function, in a neuropsychiatric patient or healthy individual.

Dementia is conventionally evaluated by a set of clinical tests applied by trained physicians and certified neuropsychologists. Professionals primarily use screening tests such as Mini-Mental Status Examination (MMSE) and Montreal Cognitive Assessment (MoCA), or more detailed tests such as Adenbrooke's Cognitive Examination, ADAS-Cog, and Blessed orientation memory concentration test.

Alzheimer's disease (AD) is a most debilitating neurodegenerative disorder with a most significant burden on western society. AD is an impending epidemic, plaguing the Baby Boomer generation and causing immeasurable suffering to patients and families. AD is currently diagnosed as based on the combination of general cognitive deterioration with deficits in memory and another cognitive domain. Despite extensive research the core cognitive deficit in AD is still unknown.

### SUMMARY OF THE INVENTION

Conventional tests for evaluating dementia and AD are time consuming in the framework of the Emergency Room, clinical ward or a busy clinic. It was realized by the Inventors of the present invention that conventional tests such as Addenbrooke's Cognitive Examination and Blessed orientation memory concentration test, are static in the sense that they allow measuring success rates, but not strategies, timings or dynamics. Conventionally, patients are evaluated through a prolonged neuropsychological testing which is long and costly, examiner dependent and/or inaccurate. The present inventors realized that such a low-tech test does not offer dynamic measurements, and that complicated tasks that may be quantified in many cases are scored on a binary score, which in many cases is ill-defined.

Some embodiments of the present invention are based on the impairment of mental orientation.

As used herein, a mental orientation of individual refers to a cognitive function that reflects the awareness of the individual with respect to at least one of, more preferably at least two of, more preferably each of, time (events), person (people) and space (places).

The mental orientation thus processes the relations between the behaving self to space, time, and person.

The present inventors have successfully characterized the mental orientation cognitive function and discovered the underlying brain system. The present inventors clinically established the relations between mental orientation and Alzheimer's disease and found that the mental orientation relates to brain regions disturbed in AD and other several specific neurological disorder as measured by both functional and structural modes. The present inventors have demonstrated that mental orientation is a distinct cognitive function, which determines one's self-reference to a cognitive map of landmarks in space (places), time (events), and person (people) and is based on shared cognitive and neural mechanisms.

The present inventors successfully demonstrate that the determination of a mental orientation allows assessing Alzheimer's disease. The present inventors found that the neural network underlying orientation overlaps with brain regions affected in Alzheimer's disease.

The present inventors have devised a mental task that can optionally and preferably be used, in combination with functional neuroimaging, to characterize mental orientation as well as its underlying network of interacting brain regions. The mental task can also be supplemented by additional neuropsychological tests to diagnose specific types of cognitive decline.

The present inventors have optionally and preferably also devised a system that optionally and preferably analyzes the events, places and people (EPPs) that are specific to the individual, create a subject-specific task that can optionally and preferably be used to characterize mental orientation. The system and method of the present embodiments are optionally and preferably adapted to patients along the AD spectrum and optionally also one or more other cognitive and neuropsychiatric disorders. The present inventors discovered norms, patterns and signatures of AD and other cognitive disorders and some embodiments of the present invention exploit these norms, patterns and/or signatures for assessing the cognitive function of a subject. Some embodiments of the present invention provide a system and a method to support and improve or maintain mental orientation of a subject.

The present embodiments can thus be used as a platform for assessing cognitive decline including a wide spectrum of AD, and is therefore useful for individuals, families, caregivers and healthcare professionals. The platform of the present embodiments can identify cognitive deterioration before significant impairment to the brain occurs and can allow users to maintain orientation based on their digital footprint and assessment.

The mental subject-specific cognitive task of the present embodiments optionally and preferably provides individually tailored stimuli in at least one domain selected from the group consisting of space (places), time (events) and person (people). The task may be in the form of a set of subject-specific questions. In some embodiments of the present invention the response of the subject to each of these questions is evaluated automatically by a data processor, and is analyzed based on norms, patterns and/or signatures that are obtained from a computer readable memory medium and that allow the data processor to characterize different dementias of the present embodiments relying, in part, on additional computerized cognitive task. The system of the present embodiments can optionally and preferably include one or several modules, including without limitation, at least one of a module for computerized cognitive assessment, a machine learning module and a neurophysiological data module.

According to an aspect of some embodiments of the present invention there is provided a method of neuropsychological analysis. The method comprises: presenting to a subject, by a user interface, a subject-specific cognitive task having at least one task portion selected from the group consisting of a time-domain task portion, a space-domain task portion, and a person-domain task portion. The method also comprises receiving responses entered by the subject using the user interface for each of the task portions, representing the responses as a set of parameters, and classifying the subject into one of a plurality of cognitive function classification groups, based on the set of parameters.

According to some embodiments of the invention the subject-specific cognitive task comprises at least two of the time-domain, space-domain and person-domain task portions.

According to some embodiments of the invention the subject-specific cognitive task comprises each of the time-domain, space-domain and person-domain task portions.

According to some embodiments of the invention the method comprises constructing the subject-specific cognitive task.

According to some embodiments of the invention the method comprises presenting a questionnaire to an individual other than the subject and receiving a response to the questionnaire, wherein the subject-specific cognitive task is constructed based on the response to the questionnaire.

According to some embodiments of the invention the constructing the subject-specific cognitive task is executed automatically.

According to some embodiments of the invention the method comprises receiving from a mobile device of the subject sensor data, wherein the subject-specific cognitive task is constructed based on the sensor data.

According to some embodiments of the invention the method comprises accessing a social network account associated with the subject, and extracting social interaction data from the account, wherein the subject-specific cognitive task is constructed based on the social interaction data.

According to some embodiments of the invention the method comprises receiving from a mobile device of the subject stored social interaction media, wherein the subject-specific cognitive task is constructed based on the stored social interaction media.

According to some embodiments of the invention the subject-specific cognitive task is constructed using a machine learning process.

According to some embodiments of the invention the method comprises receiving from a mobile device of the subject sensor data, wherein the classification is based also on the sensor data.

According to some embodiments of the invention the sensor data comprise data selected from the group consisting of location data, acceleration data, orientation data, audio data and imaging data.

According to some embodiments of the invention the mobile device comprises a touch screen and the sensor data comprise data selected from the group consisting of touch pressure data, and touch duration data.

According to some embodiments of the invention the method comprises scoring the classification.

According to some embodiments of the invention the method comprises transmitting the classification to a remote location over a communication network.

According to some embodiments of the invention the method comprises receiving from a neurophysiological data acquisition system neurophysiological data pertaining to a brain of the subject, wherein the classification is based also on the neurophysiological data.

According to some embodiments of the invention the method comprises accessing a library of reference data comprising at least parameters describing responses of previously classified subjects, and processing and analyzing the set of parameters using at least a portion of the reference parameters, wherein the classification is based also on the analysis.

According to some embodiments of the invention the processing comprises applying a machine learning process.

According to some embodiments of the invention the machine learning procedure comprises a supervised learning procedure.

According to some embodiments of the invention the machine learning procedure comprises at least one procedure selected from the group consisting of clustering, support vector machine, linear modeling, k-nearest neighbors analysis, decision tree learning, ensemble learning procedure, neural networks, probabilistic model, graphical model, Bayesian network, boosting, and association rule learning.

According to some embodiments of the invention the method comprises altering the cognitive task based on the responses, presenting the altered cognitive task to the subject, and receiving responses entered by the subject using the user interface for the altered cognitive task, wherein the classification is based on a comparison between responses entered before the alteration.

According to some embodiments of the invention the method comprises presenting to the subject by the user interface, a feedback pertaining to at least one of the responses.

According to some embodiments of the invention the method comprises re-presenting the cognitive task to the subject following the feedback, and receiving responses entered by the subject using the user interface for the re-presented cognitive task, wherein the classification is based on a comparison between responses entered before the feedback and responses entered after the feedback.

According to some embodiments of the invention the method comprises presenting to an individual other than the subject, information pertaining to at least one of the responses.

According to some embodiments of the invention the method comprises presenting to a subject, by a user interface, at least one additional cognitive task, and receiving a response entered by the subject for each of the at least one additional task using the user interface for the at least one additional cognitive task, wherein the classifying is based also on the response to the at least one additional cognitive task.

According to some embodiments of the invention the method comprises evaluating effects of a treatment applied to the subject for the classified cognitive function.

According to some embodiments of the invention the method comprises treating the subject for the classified cognitive function.

According to some embodiments of the invention the treatment is selected from the group consisting of pharmacological treatment, ultrasound treatment, rehabilitative treatment, electrical stimulation, magnetic stimulation, phototherapy, and hyperbaric therapy.

According to an aspect of some embodiments of the present invention there is provided a server system for neuropsychological analysis. The server system comprises: a transceiver arranged to receive and transmit information on a communication network; and a processor arranged to communicate with the transceiver, and perform code instructions. The code instructions can comprise code instructions for transmitting to a client computer, a subject-specific cognitive task to be presenting to a subject by a user interface, the cognitive task having a time-domain task portion, a space-domain task portion, and a person-domain task portion. The code instructions can also comprise code instructions for receiving from the client computer responses for each of the task portions, code instructions for representing the responses as a set of parameters, and code instructions for classifying the subject into one of a plurality of cognitive function classification groups, based on the set of parameters.

According to some embodiments of the invention the processor is arranged to perform code instructions for executing the method as delineated above and optionally and preferably exemplified below.

According to some embodiments of the invention the plurality of cognitive function classification groups comprises Mild Cognitive Impairment (MCI), Alzheimer's disease (AD), and age related cognitive decline.

According to some embodiments of the invention the classifying comprises applying a domain-specific weight to each of the parameters.

According to some embodiments of the invention the classifying comprises applying logistic regression.

According to some embodiments of the invention the classifying comprises applying ordinal logistic regression.

According to some embodiments of the invention the set of parameters comprises, for at least one of the task portions, a success rate and a response time.

According to some embodiments of the invention at least one of the task portions comprises a first stimulus, a second stimulus and an instruction to rate a level of relationship between the subject and each of the stimuli.

According to some embodiments of the invention at least two the of the task portions comprise different stimuli but similar instruction.

According to some embodiments of the invention at least one of the task portions comprises a single assignment.

According to some embodiments of the invention at least one of the task portions comprises a plurality of assignments.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard, touch-screen or mouse are optionally provided as well.

### BRIEF DESCRIPTION OF SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for neuropsychological analysis, according to some embodiments of the present invention;

FIG. 2 is a schematic illustration of a server-client configuration, according to some embodiments of the present invention;

FIG. 3 is a block diagram schematically illustrating a computation system according to some embodiments of the present invention;

FIG. 4 is a schematic illustration of relationships of a subject within different domains, according to some embodiments of the present invention;

FIGS. 5A-H show representative screen shots suitable for use according to some embodiments of the present invention;

FIGS. 6A and 6B show a global field power (FIG. 6A) and an evoked potential map of a microstate class, as obtained in experiments performed according to some embodiments of the present invention;

FIG. 7 is a schematic illustration showing data flow in an exemplified platform designed according to some embodiments of the present invention;

FIG. 8 is a schematic illustration showing a more detailed data flow in an exemplified platform designed according to some embodiments of the present invention;

FIG. 9 is a flowchart diagram showing a representative protocol according to some embodiments of the present invention;

FIGS. 10A-E show behavioral results, obtained in experiments performed according to some embodiments of the present invention;

FIGS. 11A-E show age and education comparable subsets, obtained in experiments performed according to some embodiments of the present invention;

FIGS. 12A-D show success rate and response time analyses, obtained in experiments performed according to some embodiments of the present invention;

FIGS. 13A-D show machine-learning based analyses, obtained in experiments performed according to some embodiments of the present invention;

FIGS. 14A-D show evoked brain activity, obtained in experiments performed according to some embodiments of the present invention;

FIGS. 15A-D show time, space, person and default network overlap, obtained in experiments performed according to some embodiments of the present invention;

FIGS. 16A-D show midsagittal cortical activity during orientation in space, time, and person, obtained in experiments performed according to some embodiments of the present invention;

FIGS. 17A-D show lateral cortical activity during orientation in space, time, and person, obtained in experiments performed according to some embodiments of the present invention;

FIG. 18 shows cortical activity during orientation in space, time, and person in 16 individual subjects, obtained in experiments performed according to some embodiments of the present invention;

FIG. 19 shows overlap between activations in the different orientation domains in 16 individual subjects, obtained in experiments performed according to some embodiments of the present invention;

FIGS. 20A-B show random-effects group analysis, obtained in experiments performed according to some embodiments of the present invention;

FIGS. 21A-B show probabilistic-maps group analysis, obtained in experiments performed according to some embodiments of the present invention;

FIG. 22 shows overlap between default-mode network and activity during orientation in a person domain for 14 individual subjects, obtained in experiments performed according to some embodiments of the present invention;

FIG. 23 shows overlap between default-mode network and activity during orientation in a space domain for 14 individual subjects, obtained in experiments performed according to some embodiments of the present invention;

FIG. 24 shows overlap between the default-mode network and activity during orientation in a time domain for 14 individual subjects, obtained in experiments performed according to some embodiments of the present invention;

FIG. 25 shows average default-mode network overlap with orientation domains for individual subjects, obtained in experiments performed according to some embodiments of the present invention;

FIGS. 26A-C show event-related time courses from default-mode networks nodes, for the different orientation domains, obtained in experiments performed according to some embodiments of the present invention;

FIGS. 27A-B show overlap between activations in the space, time, and person domains, obtained in experiments performed according to some embodiments of the present invention;

FIGS. 28A-C show overlap of orientation activity with the default mode network, obtained in experiments performed according to some embodiments of the present invention;

FIG. 29 is a representative examples of stimuli presented to subjects in experiments performed according to some embodiments of the present invention;

FIGS. 30A-C show EP mapping of young healthy subjects, obtained in an experiment that was performed according to some embodiments of the present invention and that included young healthy subjects;

FIGS. 31A-E show results obtained in an experiment that was performed according to some embodiments of the present invention and that included patients along the AD-spectrum; and

FIGS. 32A-D show mean reaction times and efficiency scores, as obtained in experiments performed according to some embodiments of the present invention.

### DESCRIPTION OF SPECIFIC EMBODIMENTS OF THE INVENTION

The present invention, in some embodiments thereof, relates to neuromedicine and, more particularly, but not exclusively, to a method and system for assessing a cognitive function, in a neuropsychiatric patient or healthy individual.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

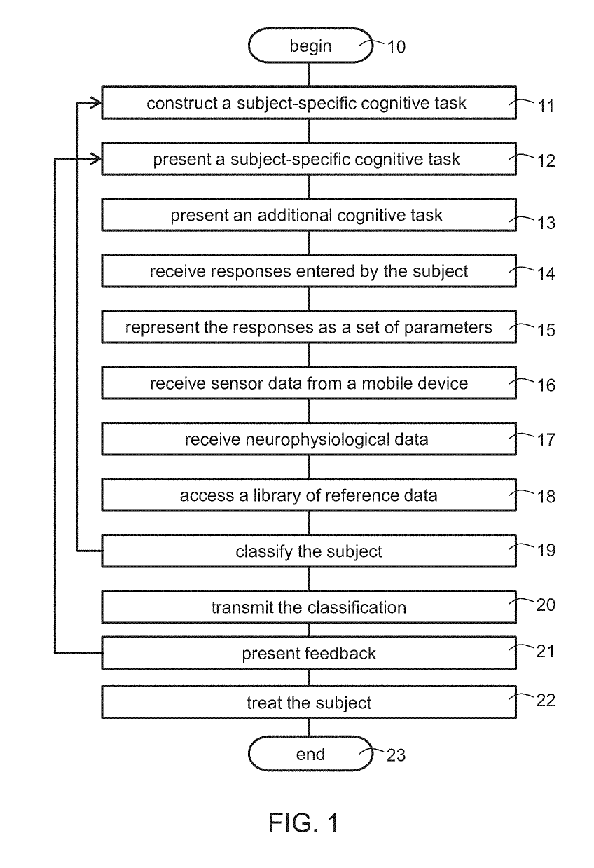

FIG. 1 is a flowchart diagram of a method suitable for neuropsychological analysis, according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be can be implemented by a data processing system, e.g., a dedicated circuitry or a general purpose computer, configured for receiving data and executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

The method of the present embodiments can be used for assessing the cognitive function of a subject. For example, the method can be used to classify the subject into one of a plurality of cognitive function classification groups. Each cognitive function classification group can be characterized by a cognitive function or dysfunction. Representative examples of classification groups suitable for the present embodiments include, without limitation, a Mild Cognitive Impairment (MCI) classification group, an Alzheimer's disease (AD) classification group, a classification group encompassing one or more other dementias, and an age related cognitive decline classification group. Other classification groups are also contemplated. For example, two or more AD or MCI classification groups can be defined, for different severities of the AD or MCI.

Referring to FIG. 1 the method begins at 10 and optionally continues to 11 at which a subject-specific cognitive task is constructed. Representative examples for a procedure suitable for constructing a subject-specific cognitive task are provided hereinafter. The subject-specific cognitive task can alternatively be retrieved from a source such as, but not limited to, a computer-readable medium, in which case 11 can be skipped.

The subject-specific cognitive task optionally and preferably comprises one or more task portions. A task portion typically includes one or more assignments. An assignment typically includes an information section and an instruction section. In some embodiments of the present invention the information section includes two or more objects, and the instruction section includes a human language message requesting the subject to select or rate one or more of the objects in the information section of the assignment.

The cognitive task is “subject specific” in the sense that the objects in the information section of the assignments are optionally and preferably selected such that they would have been likely to be recognized by the subject, had the subject been cognitively normal.

A task portion can be a time-domain task portion. In these embodiments, the information section of an assignment of the task portion optionally and preferably describes an event, and the instruction section optionally and preferably requests the subject to rate the event in terms of the temporal distance to the event. Alternatively, the information section of an assignment of time-domain task portion can describe two or more events occurring at different times, and the instruction section can request the subject to time-order these event. For example, when information section describes two events, the instruction section can request the subject to select which of the two events occurred earlier in the past.

A task portion can be a space-domain task portion. In these embodiments, the information section of each assignment of the task portion optionally and preferably describes a place, and the instruction section optionally and preferably requests the subject to rate the spatial distance to the described place with respect to the subject's current location. Alternatively, the information section of an assignment of space-domain task portion can describe two or more places located at spaced apart locations, and the instruction section can request the subject to order these places according to their location, more preferably according to their distances with respect to the subject's current location and/or thereamongst. For example, when information section describes two places, the instruction section can request the subject to select which of the two places is farther from the subject.

A task portion can be a person-domain task portion. In these embodiments, the information section of each assignment of the task portion optionally and preferably describes a person, and the instruction section optionally and preferably requests the subject to rate the person according to his or hers social, familial or emotional proximity to the subject. Alternatively, the information section of an assignment of person-domain task portion can describe two or more persons, and the instruction section optionally and preferably requests the subject to order these persons according to their social, familial or emotional proximity to the subject and/or thereamongst. For example, when information section describes two persons, the instruction section can request the subject to select which of the two persons is closer to the subject in terms of interpersonal relationship.

In some embodiments of the subject-specific cognitive task includes at least one of the of the time-domain, space-domain and person-domain task portions, in some embodiments of the present invention the subject-specific cognitive task includes at least two of the time-domain, space-domain and person-domain task portions, and in some embodiments of the present invention the subject-specific cognitive task includes all three of the time-domain, space-domain and person-domain task portions.

The method optionally and preferably continues to 12 at which the subject is presented with the subject-specific cognitive task. The subject-specific cognitive task is optionally and preferably presented by a user interface such as, but not limited to, a graphical user interface displayed on a computer screen, a smart TV screen, or a screen of a mobile device, e.g., a smartphone device, a tablet device or a smartwatch device. The subject-specific cognitive task optionally and preferably comprises a plurality of task portions.

The task portions, or the assignment(s) thereof, are typically presented in a human-readable form to allow the subject to read and decipher them. The task portions or assignment(s) can be presented as textual objects, indicia, symbols, animations and/or images on the user interface. Combinations of two or more of these presentation forms are also contemplated. For example, a particular assignment can include an image accompanied by a textual object or an indicium. A typical example for such an assignment is an assignment of a person-domain task portion, wherein the information (person) is presented as an image and the instruction (e.g., “rate the proximity”) is presented as a text message.

Typically, but not necessarily the task portions are presented sequentially on the user interface. When a task portion includes several assignments (for example, several assignments each including an information section and an instruction section) the assignments can be presented immediately one after the other, or simultaneously on different parts of the user interface, or intermittently (for example, one or more assignments of task portion in a particular domain, can be presented between two assignments of a task portion in another domain).

In various exemplary embodiments of the invention the subject is presented also with a set of controls, preferably on the same screen as the respective task portions, to allow the subject to respond to the task portions. The controls can be presented separately or combined with other sections of the presented task. Typically, but not necessarily, the controls are combined with the information sections of the respective assignment so that the subject can easily selects the respective object (e.g., event, place, person) as a response to the assignment or task portion. In some embodiments of the present invention one or more rating controls are presented for allowing the subject to rate the object(s) displayed in the information section. The rating control can provide a scale for the rating. Optionally and preferably, the scale is a non-binary scale. The scale can be a discrete scale, having a set of discrete descriptors, optionally and preferably, a set of at least 3 or at least 4 or at least 5 or more discrete descriptors, or a continuous scale having a continuum of descriptors. A set of discrete descriptors can be an ordinal set of integer numbers, or a set of human language descriptors (e.g., “do not agree at all,” “agree”, “very much agree”, “do not know”). A continuum of descriptors can include a continuum of numbers from a minimum number (e.g., 0) to a maximum number (e.g., 5, 10, 100, etc.). The rating control of an assignment can be of any type generally known in the field of graphical user interface design. Representative examples include, without limitation, a slider, a dropdown menu, a combo box, a text box and the like.

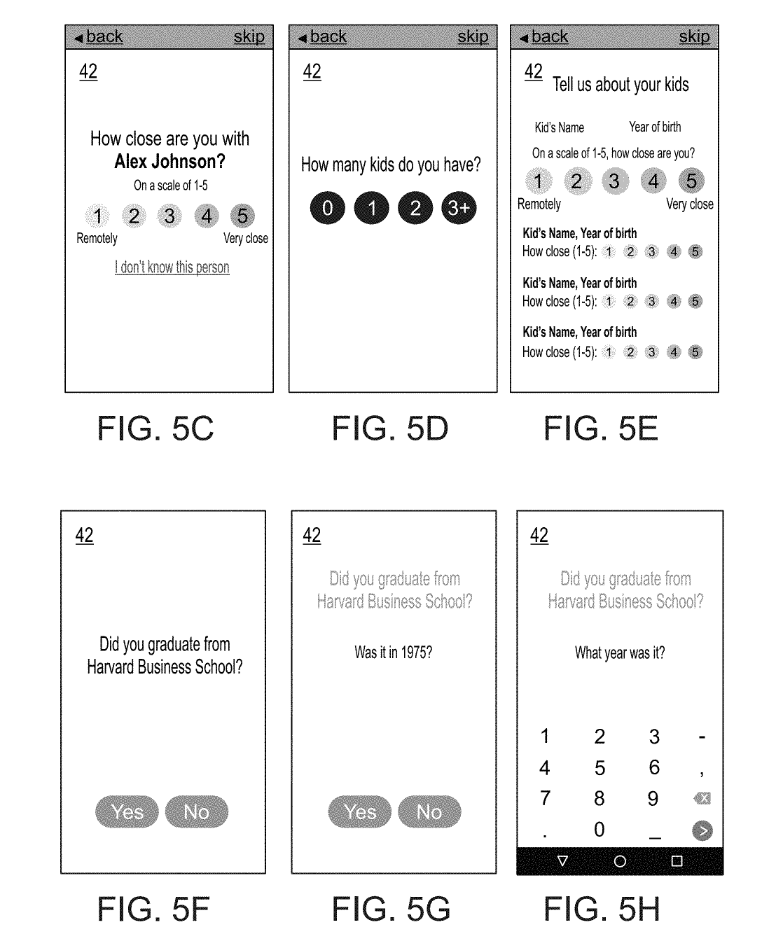

Representative examples of screen shots suitable for use as a user interface presenting the subject-specific cognitive task optionally and preferably are provided in FIGS. 5A-H.

Optionally, the method proceeds to 13 at which the subject is presented, preferably by the same user interface, an additional cognitive task. The additional cognitive task can be of any type known in the art that can cause brain activation. Representative examples include, without limitation, a recollection task, a memory task, a working memory task, an abstract reasoning task, an object recognition task, an odor recognition task, a standard-orientation test, mini-mental state examination (MMSE) and the like. In some embodiments of the present invention the additional cognitive task is non-subject-specific, in the sense that it is presented irrespectively of the subject's identity.

The method optionally and preferably continues to 14 at which responses entered by the subject using the user interface are received for each of the task portions. When an additional task is presented, the method receives at 14 also the subject's response(s) to the assignments of the additional task. In embodiments in which controls are presented, the user enters the responses using the controls, and the responses are received from the controls. Each received response optionally and preferably corresponds to one assignment presented on the user interface.

At 15 the method preferably represents the responses as a set of parameters. Typically, each response is represented as one or more parameters. For example, a response can be represented by a success parameter indicative of the correctness or accuracy of the response. A parameter indicative of the correctness of the response can be a binary parameter, or a non-binary parameter, which can be a discrete non-binary parameter or continuous non-binary parameter. The response can be alternatively or additionally be represented by a response time parameter, which can be defined as the elapsed time between the presentation of the assignment and the time at which the subject provided the response. Preferably, each response is represented by a success parameter and by a response time parameter.

In some optional embodiments of the present invention, the method proceeds to 16 at which the method receives from a mobile device of the subject sensor data, wherein classification is based also on sensor data. The mobile device can be any of a variety of portable computing devices including, without limitation, a cell phone, a smartphone, a handheld computer, a laptop computer, a notebook computer, a tablet device, a notebook, a media player, a Personal Digital Assistant (PDA), a camera, a video camera and the like. The sensor data can be received from any of the sensors of the mobile device. Representative examples of sensor data that can be received at 16 include, without limitation, accelerometeric data, gravitational data, gyroscopic data, compass data, GPS geolocation data, proximity data, illumination data, audio data, video data, temperature data, geomagnetic field data, orientation data, imaging data and humidity data. When the mobile device comprises a touch screen, the sensor data optionally and preferably comprises touch pressure data and/or touch duration data.

In some optional embodiments of the present invention, the method proceeds to 16 at which the method receives from a neurophysiological data acquisition system neurophysiological data pertaining to the brain of the subject. The neurophysiological data acquisition system can be of any type capable of receiving signals from the brain.

Preferably, the system is an electroencephalogram (EEG) system including a plurality of electrodes placeable on the scalp of the subject. Other systems that are contemplated according to some embodiments of the present invention include, without limitation, magnetoencephalography (MEG) system, computer-aided tomography (CAT) system, positron emission tomography (PET) system, magnetic resonance imaging (MRI) system, functional MRI (fMRI) system, Near infra red system (NIRS), ultrasound system, single photon emission computed tomography (SPECT) system, and Brain Computer Interface (BCI) system.

At 19 the subject is optionally and preferably classified into one of a plurality of cognitive function classification groups. Optionally, the classification is accompanied by a score which is indicative of the likelihood that the subject is a member of the respective classification group. Optionally, the classification is transmitted 20 to a computer readable medium and/or a display device. The computer readable medium and/or display device can be local with respect to the computer that performs the classification. Alternatively, or additionally, the classification can be transmitted 20 to a computer readable medium and/or a display device at a remote location, for example, at a client computer (e.g., of a clinician or another individual or the subject). The classification is preferably based at least on the set of parameters provided at 15. As demonstrated in the Examples section that follows, parameters representing responses to time-domain, space-domain and/or person-domain task portions provide information regarding the mental orientation of the subject and can therefore be used for discriminating between different types and levels of cognitive dysfunction. It was found that the use of these types of parameters allows classifying the subject with improved accuracy compared to other techniques. In a comparative set of experiments performed by the Inventor (data not shown) the classification accuracy was about 95% when using the method according to some embodiments of the present invention, and 74% when using the Addenbrooke's Cognitive Examination.

In some embodiments, the classification is executed using a classifier, such as, but not limited to, a logistic regression function, an ordinal logistic regression function, a decision tree, a support vector machine (SVM), a maximum entropy function, etc. In these embodiments, the set of parameters is fed into the classifier to provide a score. The score can be compared to one or more predetermined thresholds and the subject can be classified based on the comparison. A single threshold can be used for double classification. For example, for a subject suspected as (e.g., previously diagnosed) having cognitive dysfunction, when the score is above the threshold the subject is classified as having an age related cognitive decline, and when the score is below the threshold the subject is classified as having AD or MCI. Two thresholds can be used for double classification. For example, for a subject suspected as having cognitive dysfunction, when the score is above both thresholds the subject is classified as having an age related cognitive decline, when the score is between the thresholds the subject is classified as having MCI and when the score is below both thresholds the subject is classified as having AD.

When the subject is presented with an additional cognitive task, the response(s) to this task are optionally and preferably also used for the classification. These embodiments are particularly useful when it is desired to improve the specificity of the classification. For example, when a particular additional task is known to discriminate between two classification groups or classification subgroups, a combined score can be computed based on the parameters that represent the subject-specific task as well as the parameter(s) that represent the additional task, and the combined score can be utilized for the classification, for example, by thresholding as further detailed hereinabove. The combined score can optionally and preferably be computed by a machine learning process, optionally and preferably a previously trained machine learning process, which receives parameters representing the responses as input and provide a combined score as output.

The classification can optionally and preferably be also based on the neurophysiological data (in embodiments in which such data are collected). In these embodiments, the method optionally and preferably searches for patterns in the data that are indicative of a particular cognitive dysfunction.

The present inventors found that the use of EEG recorded during performance of the subject-specific cognitive task allows detecting of orientation and its disorders.

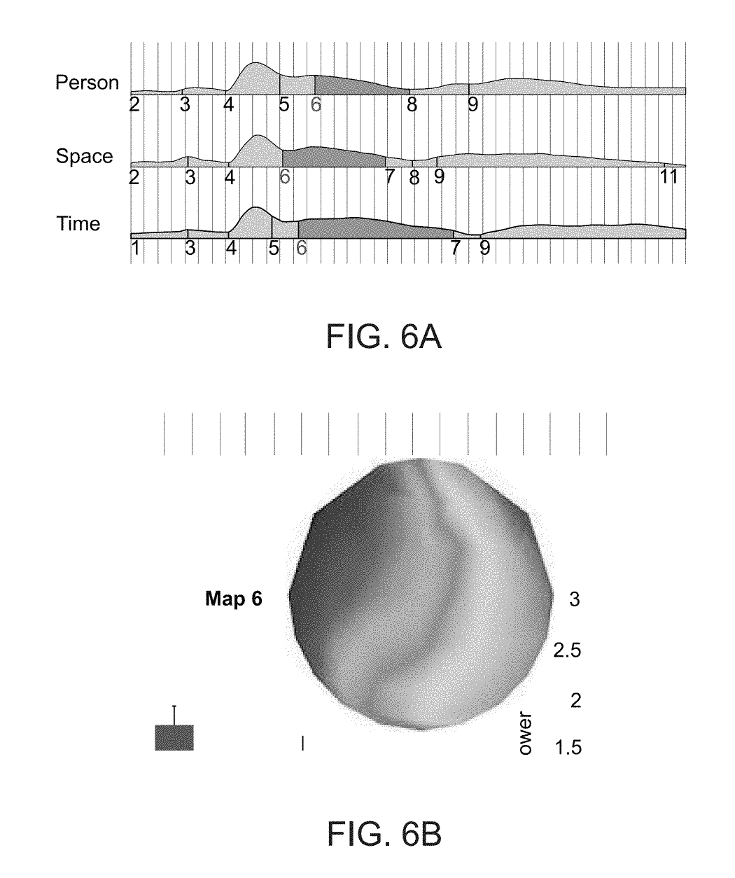

It was specifically found by the inventors that EEG data can be used to construct a signature that is specific to the subject's cognitive function, and is optionally and preferably also specific to the domain of the task portion. This signature can represent the global electrical field produced by the brain during one or more cognitive and mental activities. It was specifically found that EEG data obtained during the presentation of each task portions (in the time-, space- and person-domains) are distinguished from EEG data obtained in the absence of task portion presentations. Such a distinction can be realized by constructing microstate maps of the subject's brain from the EEG data. Representative examples of microstate maps that can serve as signatures according to some embodiments of the present invention are shown in FIGS. 6A-B. Shown in FIGS. 6A and 6B are results of experiments in which a multi-channel (64 electrodes) EEG was recorded while 14 young healthy subjects were presented with the subject-specific task optionally and preferably. The EEG data were processed by cluster analysis to define brain microstates and generate a series of Evoked Potential (EP) maps, each corresponding to a class of microstates and describing a different spatial distribution of electric potential over the brain. Each map was assigned with a serial class number. A global field power, which is a parametric assessment of the strength of each EP map, was also calculated by computing deviations of momentary potential values.

FIG. 6A shows the global field power over a time axis. Shown are time segments at which each class of EP maps appeared. The time axis in FIG. 6A is divided to ˜100 ms epoches, and the serial class numbers of the respective maps are indicated on the axis. Thus, during an experiment in which the person-domain task portion was presented, EP maps of class No. 2 appeared over the first four epochs, EP maps of class No. 3 appeared over epoch Nos. 5-7, EP maps of class No. 4 appeared over epoch Nos. 8-11, and so on.

It was surprisingly and unexpectedly found by the Inventors that maps belonging to a microstate class showing a gradient gradually evolving from the right posterior parietal cortex to the left inferior frontal cortex, distinguished EEG data acquired during presentation of any of the task portions of the present embodiments from EEG data acquired otherwise. FIG. 6B shows, in color codes, exemplary EP map of such a microstate class. Additional EP maps are provided in the Examples section that follows.

Thus, according to some embodiments of the present invention the EEG data acquired from the subject is analyzed to determine whether a particular class of EP maps, such as a class showing a gradient gradually evolving from the right posterior parietal cortex to the left inferior frontal cortex, exists in the data, and the classification of the subject is based on this analysis. For example, when the particular class of EP maps does not exist in the data or is altered, the method can accord more weight to the probability that the subject has a cognitive dysfunction.

The present inventors found that a microstate class with a gradient gradually evolving from the right posterior parietal cortex to the left inferior frontal cortex appears longer and stronger for the time-domain task portion than to the person-domain and spatial-domain task portions, and was absent in control tasks. This map can therefore represent brain activity related to orientation. Consequently, this brain state, and thus the resulted EP map, is altered in subjects with orientation disturbance, such as subjects on the AD spectrum. The map is detectable, and can thus serve as a biomarker for cognitive disturbances of orientation such as in Alzheimer's disease.

For example, an indication that a subject has Alzheimer's disease can be obtained when the time scale of the map is less than a first predetermined threshold, an indication that a subject has MCI can be obtained when the time scale of the map is less than a second predetermined threshold, and an indication that a subject has age related cognitive decline can be obtained when the time scale of the map is more that than the second predetermined threshold, wherein the second predetermined threshold is longer than the first predetermined threshold.

Another type of data that can be used is PET scan data. It was also found by the Inventors that brains PET scans show enhanced activity in the precuneus during the presentation of the task portion, wherein for subjects having MCI and AD the activity is significantly reduced. Thus, the amount of activity in the precuneus can be used as a biomarker for cognitive disturbances of orientation such as in Alzheimer's disease.

The existence, absence or extent of distinguishing patterns in the neurophysiological data (e.g., particular microstate classes, such as, but not limited to, the microstate class shown in FIG. 6B) can optionally and preferably be used, together with the other parameters to update the score and the updated score can be utilized for the classification, for example, by thresholding as further detailed hereinabove. The score can optionally and preferably be updated by a machine learning process, optionally and preferably a previously trained machine learning process, which receives the set of parameters and/or neurophysiological data as input, and provides the updated score as output. The method can optionally and preferably use the neurophysiological data for characterizing a network of interacting brain regions that underline the subject's response to one or more, preferably all, the task portions.

In embodiments in which sensor data received at 16, the sensor data are optionally and preferably used for the classification. In these embodiments, the sensor data are analyzed to provide one or more behavioral characteristics associated with the subject. Representative examples of behavioral characteristics that can be estimated include, without limitation, tone of voice, amplitude of voice, variations in amplitude and pitch, motion characteristics, volume of activity over a communication network (voice call, internet, social networks), applied pressure on a touch screen, duration of pressure on the touch screen, face expression, sweating, shaking, respiration rate, skin conductance, galvanic measurements, and sympathetic arousal. For example, voice data can be used for identifying voice changes that may signify deterioration, and/or EPPs. Voice analysis may also identify individuals interacting with the subject.

The behavioral characteristic(s) are optionally and preferably used for updating the score and the updated score can be utilized for the classification, for example, by thresholding as further detailed hereinabove. The score can optionally and preferably be updated by a machine learning process, optionally and preferably a previously trained machine learning process, which receives the set of parameters and/or behavioral characteristics as input, and provides the updated score as output.

In some embodiments of the present invention, the classification is based also on prior classifications of the subject and/or other subjects, and/or on parameters previously collected for the subject and/or other subjects. In these embodiments, the method optionally and preferably accesses 18 a library of reference data. The library can be stored in a computer readable medium, typically at a remote location, such as, but not limited to, a cloud storage facility or the like. The reference data can include reference parameters previously collected from the same subject and/or other subjects in response to subject-specific and/or additional tasks. The reference data can include reference sensor data previously collected from mobile devices of the same subject and/or other subjects. The reference data can include reference neurophysiological data previously collected by one neurophysiological data acquisition systems from the brain of the subject and/or other subjects. The reference data can include reference classification data corresponding to the reference data.

The reference data can then be processed and analyzed, together with the current data of the subject (as obtained at 15 and/or 16 and/or 17) using big data analysis techniques. For example, the reference data can be processed by applying a machine learning process, optionally and preferably a previously trained machine learning process, which receives the data, and provides the updated score as output.

The machine learning process can be a supervised or unsupervised learning procedure. Representative examples of machine learning procedures suitable for the present embodiments including, without limitation, clustering, support vector machine, linear modeling, k-nearest neighbors analysis, decision tree learning, ensemble learning procedure, neural networks, probabilistic model, graphical model, Bayesian network, and association rule learning.

Once the reference data are processed and analyzed, the score can be updated and be utilized for the classification, for example, by thresholding as further detailed hereinabove.

In some embodiments of the present invention the method loops back to 11 to alter the subject-specific task, based on any of the data obtained by the method, particularly the response received at 14. The loop back is shown from 19 but can be executed following any operation of the method. The method can then receive responses entered by the subject for the altered cognitive task, and compare that responses entered before and after the alteration. This comparison can optionally and preferably be also used for the classification. For example, when the responses are inconsistent the method can accord more weight to the probability that the subject has a cognitive dysfunction.

In some embodiments of the present invention the method proceeds to 21 at which the method present to subject, by the user interface, a feedback pertaining to one or more of responses. The advantage of this embodiment is that it aid the subject in determining the accuracy and/or correctness of the response, thereby reducing, at least temporarily, his or hers cognitive decline. Optionally, the method loops back to 12 and re-presents the subject-specific task to the subject, following the feedback. The method can receive responses entered by the subject for the re-presented cognitive task, and compare between responses entered before the feedback and responses entered after the feedback. This comparison can optionally and preferably be also used for the classification. For example, when the responses are improved following the feedback the method can accord less weight to the probability that the subject has a cognitive dysfunction.

In some embodiments of the present invention the method continues to 22 at which the subject is treated for the classified cognitive function. As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. The present embodiments contemplate any type of treatment known in the art that can abrogate, substantially inhibit, slow or reverse the progression of cognitive dysfunction. Representative examples of treatments suitable for the present embodiments include, without limitation, pharmacological treatment, ultrasound treatment, rehabilitative treatment, electrical stimulation, magnetic stimulation, phototherapy, and hyperbaric therapy.

The method ends at 23.

The subject-specific cognitive task of the present embodiments can be constructed in more than one way. Typically, but not necessarily, the subject-specific cognitive task is constructed automatically, for example, by a data processor. In some embodiments of the present invention a questionnaire is presented to an individual other than the subject, for example, using a user interface as further detailed hereinabove, and a response to the questionnaire is received. The subject-specific cognitive task can then be constructed based on the response to the questionnaire. The questionnaire can include questions pertaining to the time, space and person domains of the subject. The individual can provide events, places and persons that are familiar to the subject and the assignments can be constructed based on this information.

In some embodiments of the present invention data pertaining to the time, space and/or person domains of the subject are collected automatically, and the subject-specific cognitive task can then be constructed based on these data, optionally and preferably by means of a machine learning process that employs one or more of the aforementioned machine learning procedures. The data can include, for example, sensor data, such as, but not limited to, location data, received from the mobile device of the subject. The data can alternatively or additionally include social interaction media (e.g., images of family, friends, colleges and/or places) that are stored on the mobile device of the subject. The data can include personal information data and/or social interaction data stored, e.g., under a social network account associated with the subject.

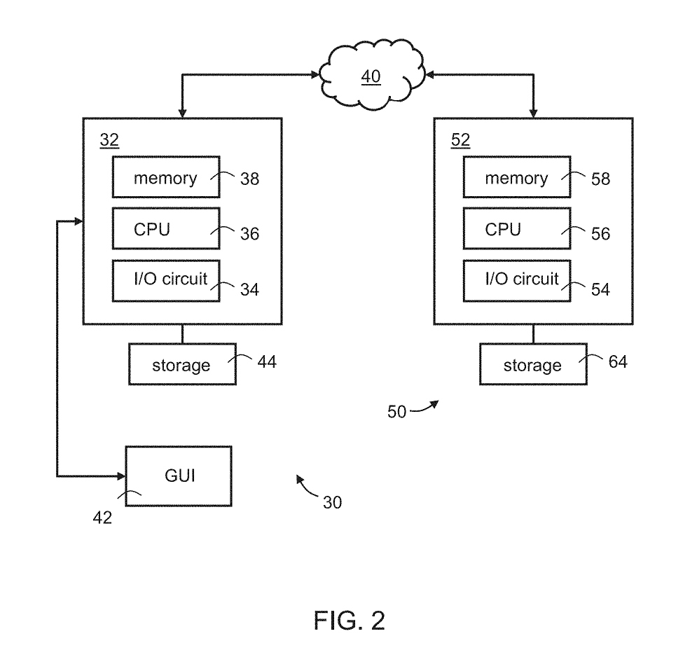

The classification of the subject according to some embodiments of the invention can be executed by a server-client configuration, as will now be explained with reference to FIG. 2.

FIG. 2 illustrates a client computer 30 having a hardware processor 32, which typically comprises an input/output (I/O) circuit 34, a hardware central processing unit (CPU) 36 (e.g., a hardware microprocessor), and a hardware memory 38 which typically includes both volatile memory and non-volatile memory. CPU 36 is in communication with I/O circuit 34 and memory 38. Client computer 30 preferably comprises a graphical user interface (GUI) 42 in communication with processor 32. I/O circuit 34 preferably communicates information in appropriately structured form to and from GUI 42. Also shown is a server computer 50 which can similarly include a hardware processor 52, an I/O circuit 54, a hardware CPU 56, a hardware memory 58. I/O circuits 34 and 54 of client 30 and server 50 computers preferable operate as transceivers that communicate information with each other via a wired or wireless communication. For example, client 30 and server 50 computers can communicate via a network 40, such as a local area network (LAN), a wide area network (WAN) or the Internet. Server computer 50 can be in some embodiments be a part of a cloud computing resource of a cloud computing facility in communication with client computer 30 over the network 40.

GUI 42 and processor 32 can be integrated together within the same housing or they can be separate units communicating with each other. GUI 42 can optionally and preferably be part of a system including a dedicated CPU and I/O circuits (not shown) to allow GUI 42 to communicate with processor 32. Processor 32 issues to GUI 42 graphical and textual output generated by CPU 36. Processor 32 also receives from GUI 42 signals pertaining to control commands generated by GUI 42 in response to user input. GUI 42 can be of any type known in the art, such as, but not limited to, a keyboard and a display, a touch screen, and the like. In preferred embodiments, GUI 42 is a GUI of a mobile device such as a smartphone, a tablet, a smartwatch and the like. When GUI 42 is a GUI of a mobile device, processor 32, the CPU circuit of the mobile device can serve as processor 32 and can execute the code instructions described herein.

Client 30 and server 50 computers can further comprise one or more computer-readable storage media 44, 64, respectively. Media 44 and 64 are preferably non-transitory storage media storing computer code instructions as further detailed herein, and processors 32 and 52 execute these code instructions. The code instructions can be run by loading the respective code instructions into the respective execution memories 38 and 58 of the respective processors 32 and 52. Storage media 64 preferably also store a library of reference data as further detailed hereinabove.

In operation, processor 32 of client computer 30 displays on GUI 42 a subject-specific cognitive task having a time-domain task portion, a space-domain task portion, and a person-domain task portion, as further detailed hereinabove. A subject, which can be suspected as having a cognitive dysfunction, enters the responses to the task portions, optionally and preferably, using controls displayed on GUI 42.

Processor 32 receives the subject's responses from GUI 42 and transmit these responses over the network 40 to server computer 50. Computer 50 receives the responses, represents the responses as a set of parameters, and classifies the subject into one of a plurality of cognitive function classification groups, based on the parameters, e.g., by computing a sore, as further detailed hereinabove. Server computer 50 can access a library of reference data, and update the score based on the reference data. Server computer 50 can receives sensor data from client computer 30, and update the score based on the reference data. Server computer 50 can also communicate with a neurophysiological data acquisition system to receive neurophysiological data pertaining to a brain of the subject therefrom and update the score based on the neurophysiological data.

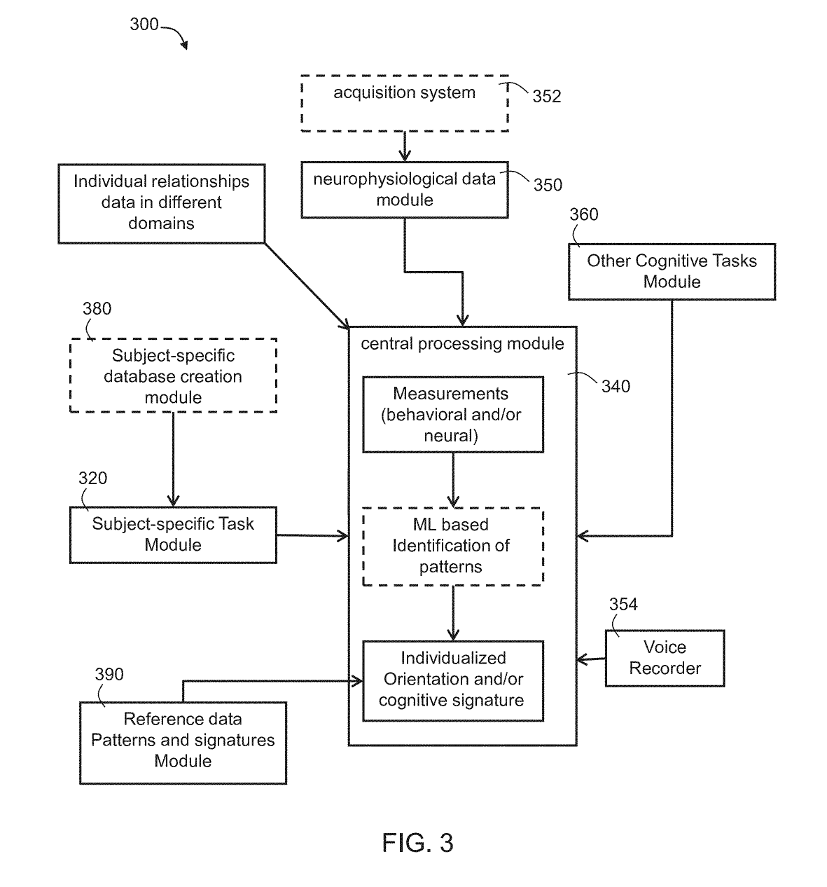

FIG. 3 is a block diagram schematically illustrating a computation system 300 that can be used for executing one or more of the operations of the method according to some embodiments of the present invention. For example, computation system 300 can be a component of server computer 50.

System 300 can be used for creating a subject-specific database that relates to the mental EPP of the subject, and/or for assessing the subject's orientation based on these EPP, and/or for assessing other cognitive domains, and/or for assessing mental orientation brain response, and/or for learning the subject-specific database, and/or for learning behavioral and/or neural patterns characterizing certain cognitive states and disorders, and/or for establishing a reference data bank of cognitive behavioral, neural measurements, patterns and/or signatures.

In some embodiments of the present invention system 300 comprises a mental subject-specific cognitive task module 320 having a circuit configured to display an subject-specific cognitive task as based on subject's EPPs collected by a subject-specific database creation module 380 (See below). The subject-specific cognitive task has different task portions, as further detailed hereinabove.

As demonstrated in the Examples section that follows, a subject-specific task having a time-domain, a space-domain, and a person-domain portions was tested in healthy volunteers and in subjects suffering from cognitive dysfunction. The subject-specific cognitive task module 320 thus preferably displays stimuli consisting of names of places (space), events (time), or people (person). The subject is optionally and preferably presented with two stimuli from the same domain (space, time, or person) and is asked to determine which of the two stimuli is closer to him or her: spatially closer to his or her current location (for space stimuli), temporally closer to the current time (for time stimuli), or personally closer to himself or herself (for person stimuli). Therefore, the task and instructions are optionally and preferably similar for each orientation domain (space, time, and person). To control for distance and difficulty effects (response-time facilitation for stimuli farther apart from each other), the subject-specific cognitive task module 320 preferably uses the subject's estimates of stimulus's distances to select pairs of stimuli with adjacent distances. Module 320 may present stimuli and collect response in any manner, including, without limitation, audio-oral manner and visuo-tactile.

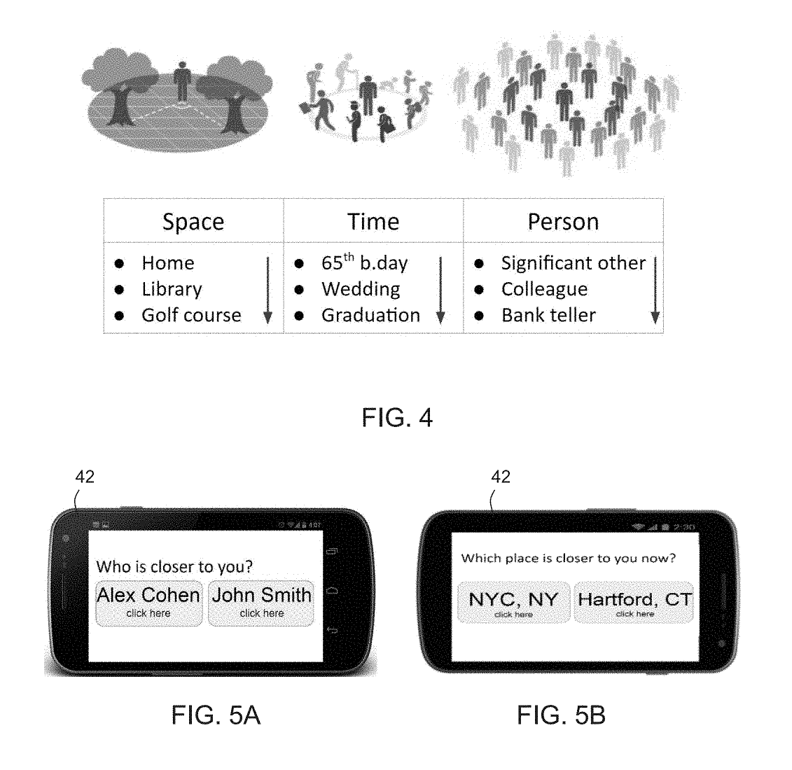

The concept of space-, time- and person-domains can be better understood from FIG. 4 which is a schematic illustration of a specific and non-limiting example of relationships the subject within the various domains. A relationship in the space domain optionally and preferably defines the proximity of the subject with different locations such as his or hers home, the library or the golf course. A relationship in the time domain optionally and preferably defines the proximity of the subject with different events such as his or hers 65^{th} birthday, a wedding and a graduation. A relationship in the person domain optionally and preferably defines the proximity of the subject with different persons such as a significant other, a colleague and his or hers bank teller.

FIGS. 5A-H schematically illustrate examples of possible assignments transmitted by the subject-specific cognitive task module of the present embodiments to a user interface such as GUI 42. The presentation of assignments and receipt of responses is referred to herein as a Digital Interviewing Process™.

In FIG. 5A, the subject is requested to choose which person is closer to him or her (an assignment of the person-domain task portion), and in FIG. 5B, the subject is requested to choose which place is closer to him or her (an assignment of the space-domain task portion). FIGS. 5A and 5B exemplify embodiments in which the response to the assignment can be represented by at least one binary parameter.

FIGS. 5C-E exemplify embodiments in which the response to the assignment can be represented by at least one discrete non-binary parameter. In FIG. 5C, the subject is requested to rate the social, familial or emotional proximity to a particular individual (displayed by name, in the present example, but can also be displayed by an image) using a 1 to 5 scale (an assignment of the person-domain task portion). The subject is also provided with the option of indicating that the displayed individual is unfamiliar to him or her. In FIG. 5D, the subject is requested to indicate the number of kids he or she has (an assignment of the person-domain task portion), and in FIG. 5E, the task portion the subject is asked about his or hers kids' name and their year of birth (a multiplicity of assignments of the person-domain task portion).

FIGS. 5F-H exemplify a series of sequentially displayed validation assignments. FIGS. 5F and 5G exemplify assignments in which the subject is requested to question by “yes” or “no” (binary response). FIG. 5H is a conditionally displayed assignment which is displayed when the subject select one binary option in the previous assignment (“no” in the present example), and is not displayed when the subject select another binary option in the previous assignment (“yes” in the present example).

Referring again to FIG. 3, system 300 optionally and preferably comprises a subject-specific database creation module 380 having a circuit configured to create the entries of the subject-specific database.

The term “subject-specific database,” as used herein refers to a database that is specific to the subject and that includes a plurality of information objects, each information object belonging to at least one domain selected from the group consisting of the time-domain, the space-domain and the person-domain, as further detailed hereinabove.

The subject-specific database of the present embodiments can includes a plurality of entries, including, without limitation, social entries, historical entries, geographical entries, clinical entries, linguistic entries, and any combination and combination of combinations thereof (e.g., socio-historical entries, socio-geographical entries, socio-geo-historical entries etc.).

Module 380 is optionally and preferably configured for collecting information regarding the subject's EPPs for use in the subject-specific task of the present embodiments. Module 380 can also be configured to employ relative closeness scale of each EPP, for pairwise comparisons in each assignment and task portion. Module 380 can also be configured to compose a multidimensional matrix representing the dynamics of the subject's mental orientation with respect to EPPs in different closeness cycle.

Module 380 is optionally and preferably configured to receive data from one or more sources (responses obtained via the Digital Interviewing Process™, sensor data from a mobile phone or wearable sensors, neurophysiological data from a neurophysiological data acquisition system, reference data from a library, data from social networks, messages composed and their digital envelope, agendas, to do lists, electronic health records, smartphone usage, computer usage, Internet activity, etc.), focusing on events, people and places. Module 380 can utilize big-data analysis technique, such as, but not limited to, machine learning. The sensors optionally and preferably collect data indicative of the relationships between the subject and environment in different domains and the machine learning process is optionally and preferably applied in order to extract significant EPPs and subject's autonomic responses to them. Module 380 optionally and preferably uses the output of the machine learning process to create a digital representation of the subject-specific database in each domain.

Module 380 optionally and preferably collected the data automatically. For example, mobile data, GPS data (e.g. regarding significant places and events), keyboard usage or created content etc. may be analyzed. The collected data can include self-reports such as digital report of past, current or future interactions with EPPs (factual as well as emotional reports), real-time indication by the subject, e.g., transmission of a signal using a dedicated application or appliance to mark significant moments. Module 380 can extract the place, event and people involved. The Digital Interviewing Process™ may include an interactive data validation and collection layer. Module 380 may optionally and preferably be configured for automatically and interactively updating and enhancing the digital representation of the subject-specific database based on the ongoing activity in the real world, digital world and tailored virtual solutions as well as subjects' response to the orientation test. Such update can be performed using machine learning processes.

The Digital Interviewing Process™ may include predefined questions per segment (language, geographical, historical, social, etc.). More specifically, the Digital Interviewing Process™ may include automated adaptive questionnaires used for validation and enhancement of data regarding EPPs, smart navigation in a tree of predefined questions (variation in content, wording, and ordering of questions) for the purpose of maximizing accuracy of responses and information gain over groups of questions, including approximation of validity of answers using response time as well as other autonomic measures, stability of answers over questionnaires taken at different times, and consistency with data extracted from social media, real world sensors and mobile devices, evaluation of an subject's status of impairment by evaluating use of self-correction and ‘repeat instructions’ options. The Digital Interviewing Process™ may also use patterns of the subject's previous interactions with the system (including previous responses, reaction times, skip/answer patterns etc.) and global patterns over the database of all subjects for optimization of the interviewing process.

The extraction of the information may use direct algorithmic, machine learning and natural language processing methods.

System 300 can also comprises a module 360 having a circuit for generating other cognitive tasks, receiving responses from these tasks and representing the responses by parameters. In some embodiments of the present invention module 360 first verifies that the subject is capable of taking the test in order to rule delirium or other physical ailments which impede cognition, for example, by presenting a questionnaire to the subject. The questionnaire may be designed to allow verifying that the subject is not alert, not tired, in good physical health (no fever, paid, Urinary Tract Infection, etc.). The questionnaire may be designed to allow verifying that the subject is not taking any drugs which may impede cognitive function (sleeping pills, anti-epilepsy, anti-stress medications, etc.).

In some embodiments of the present invention system 300 comprises a neurophysiological data 350 that supplies neurophysiological data from a neurophysiological data acquisition system 352, such as, but not limited to, EEG, functional MRI or the like. As demonstrated in the Examples section that follows (see Examples 2 and 3), the present Inventors used fMRI to unravel the brain organization. The present Inventors successfully demonstrated activation patterns indicative of domain-specific activity in subjects. The present Inventors successfully demonstrated in neuroanatomic and schematic manners how the orientation system contains on the one hand core regions for orientation in general and specialized regions to process space, time and person on the other. These findings demonstrate a pattern of activation for orientation both generally and in a domain-specific manner. Thus, in addition to the behavioral data obtained by modules 320, 380 and 360, data obtained by module 350 can be used for the detection of orientation and its disorders (disorientation).

While the embodiments above were described with a particular emphasis to fMRI, it is to be understood that it is not necessary for acquisition system 352 to be an MRI system. The present inventors found that the use of EEG recorded during performance of the subject-specific cognitive task allows detecting of orientation and its disorders. It was specifically found by the inventors that EEG data can be used to construct a signature that is specific to the subject's cognitive function, and that is optionally and preferably also domain-specific. It was specifically found that EEG data obtained during the presentation of each task portions (in the time-, space- and person-domains) are distinguished from EEG data obtained in the absence of task portions. Representative examples of such signatures are shown in FIGS. 6A and 6B, described above.

System 300 may also comprise a reference data patterns and signatures module 390 having a circuit configured for collecting reference data. The reference data is optionally and preferably collected from multiple subjects, and may include any type of data described herein, including, without limitation, previous classifications, responses to subject-specific cognitive tasks, responses to additional cognitive tasks, clinical data, sensor data, neurophysiological data, and the like. The data can be composed out of external data, data supplied by one's internal milieu (expressed by autonomic measurements including vocal, tactile, visual and more), test's results and longitudinal analyses, user's remarks and review process. Module 390 optionally and preferably employs a machine learning process to extract informative patterns regarding interactions with EPPs, internal and external factors that affect variations in EPPs.

In various exemplary embodiments of the invention system 300 comprises a central processing module 340 having a circuit that processes outputs collected from the other modules. Although processing module 340 is shown in FIG. 3, by way of example, as a separate unit from the subject-specific cognitive task module 320 and the subject-specific database creation module 380, some or all of the processing functions of processing module 340 may be performed by suitable dedicated circuitry within the housing of the subject-specific cognitive task module 320 and/or the housing of the subject-specific database creation module 380 or otherwise associated with the subject-specific cognitive task module 320 and/or the subject-specific database creation module 380. Module 340 can uses a machine learning process to learn how variations of single EPP on the relative closeness scale affects accuracy and stability of responses to orientation questions. Module 340 can evaluate the results of the subject-specific task according to the consistency of answers, response times and autonomic responses recorded during the presentation of the task.

Module 340 can identify informative features and patterns over these features that characterize interactions with significant people and use them to identify additional significant people, variations in personal closeness to significant people and possible causes for such variations. Module 340 can identify internal and external factors that affect variations in personal closeness to significant people (either local or global trends over all relationships) and/or affect the acquired significance of places and events. Module 340 can also identify the value of each EPP on a relative closeness scale, and predict future dynamics and trajectories of interaction patterns with significant people. Module 340 can analyses data on a single subject level along time, as well as by comparison of different subject by cross sections of the extracted data. The cross sections can be according to any parameter of the data, including, without limitation, age, location, gender, marital status, number of kids, emotional states, internal dynamics, patterns of behavior, and the like.

As used herein, “exemplary” means “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find support in the following examples.

### EXAMPLES

Reference is now made to the following examples, which together with the above descriptions illustrate some embodiments of the invention in a non limiting fashion.

### Example I

### Exemplary System Design

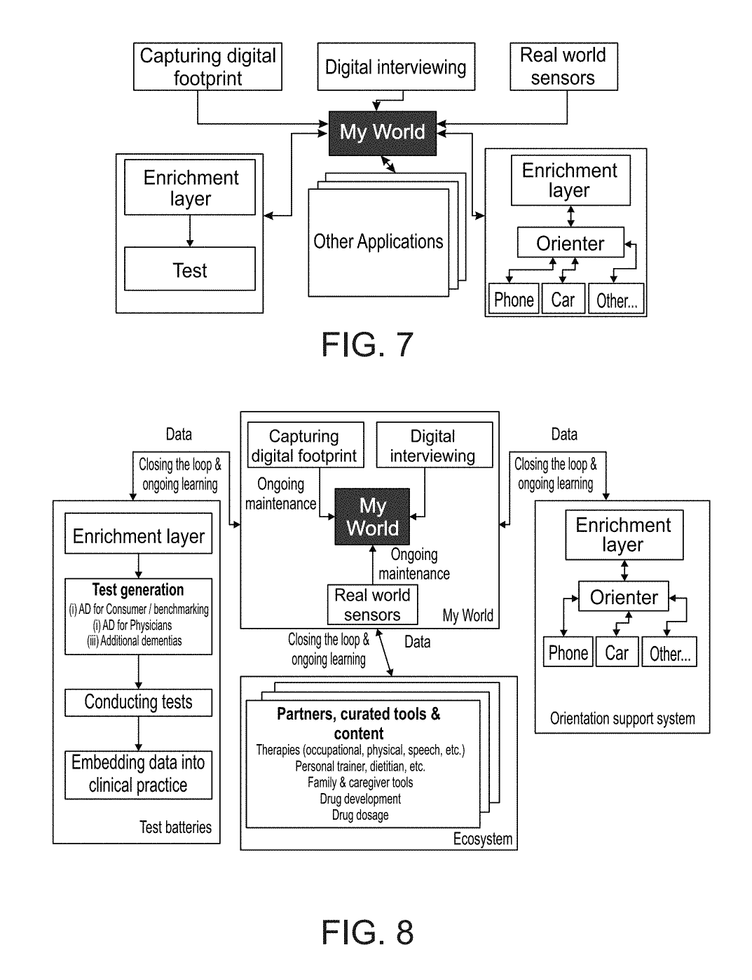

In an exemplary embodiment a digital assessment and management platform is designed. The platform optionally and preferably retrieves the subject-specific database including events, people and places in his or her life, based on data collection and analysis of the subject's digital footprint including social networks activity, use of smartphone and other hardware (wearables, IoT, etc.), created content etc, based on the Digital Interviewing Process™ of the present embodiments, and based on additional sources (electronic health records and other). The platform optionally and preferably creates a digital representation of the subject-specific database. A representative example for data flow of the platform is illustrated in FIG. 7.

The platform optionally conducts a personalized, digital assessment of the subject's orientation and cognitive systems to assess early stages of Alzheimer's disease and other dementias using the subject's personal device and/or other methods. For example, the platform can establish a cognitive baseline, screening and ongoing assessment, digitize, standardize and improve clinical assessment of cognitive functions (executive functions, language and speech, visuo-spatial, praxis, memory etc.), perform cognitive testing by a computational and touch-screen approach.

The platform optionally manages, holistically, content and partners to support patients from normal aging to early Alzheimer's disease/dementia and keeps subjects better oriented. This can be done by tools, content and APIs for (i) consumer: patients, families, and caregivers (ii) medical community: physicians, therapists (e.g., occupational therapists, physical therapists, speech therapists) ER team, etc., (iii) healthcare community (drug developers, payers, etc.), and (iv) other service providers. The platform optionally provides support for daily activities, refers to physicians and/or other therapies as needed, presents a quantified self (people, places and events) for enhancing mental-orientation to the subject's most immediate and significant environment, and identify early-stage patients for clinical trials and monitoring.

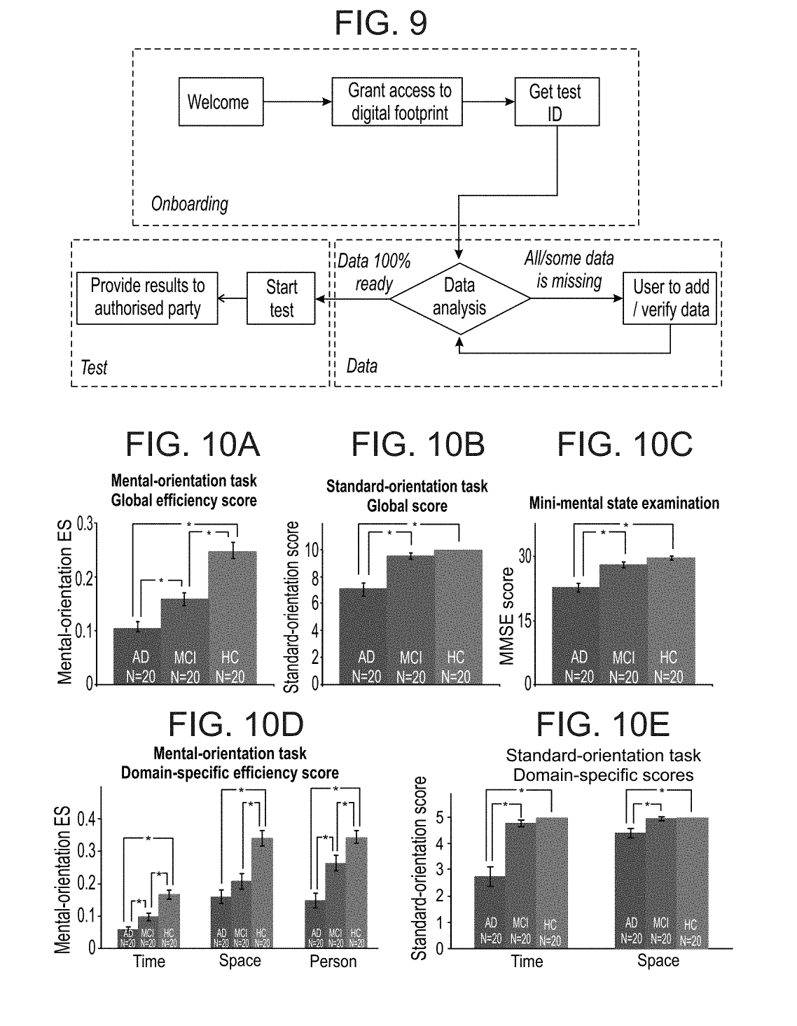

A more detailed data flow of the platform according to some embodiments of the present invention is illustrated in FIG. 8.

A system designated “My World” provides AI-based, evolving digital representation of the subject-specific database, captures the individual's digital footprint (automatic data collection infrastructure), creates a representation of the subject-specific database, focusing on events, people and places and their significance from available digital resources, collecting data from real world sensors, provides Digital Interviewing Process™, and ensures continuous maintenance by automatically and interactively updating and enhancing the digital representation of the subject-specific database based on the ongoing activity in the real world, digital world and tailored virtual solutions.

A test batteries system assesses AD spectrum and other dementias and provides automated test generation. The testing method is optionally and preferably a tablet or phone based cognitive assessment of various high-order cognitive functions. The findings are optionally and preferably embedded into clinical practice to be used by clinicians and the network of healthcare professionals.

An orientation support system supports and optionally improves the disrupted faculty, that is mental-orientation in order to help patients orient themselves and potentially slow disease progression. The orientation support system also enhances existing solutions, from neurological treatment (such as awareness of comorbidities, drug prescription and dosage) to supplementary therapies (such as speech therapy), to additional tools (such as personal training), by effectively providing orientation-related and other information.

The platform also supports back mechanisms for system's improvement, based on machine-learning analyses of the subject's status with respect to the subject-specific database, the test results and data from other applications. By combining the digital representation of the subject-specific database with a computerized dynamic test, and applying machine learning process on the data, the platform of the present embodiments can better characterize AD, its subtypes and other dementias, initiate early appropriate patient-tailored treatment, direct cognitive rehabilitation efforts and address the patient's needs along the different stages of AD and other dementias.

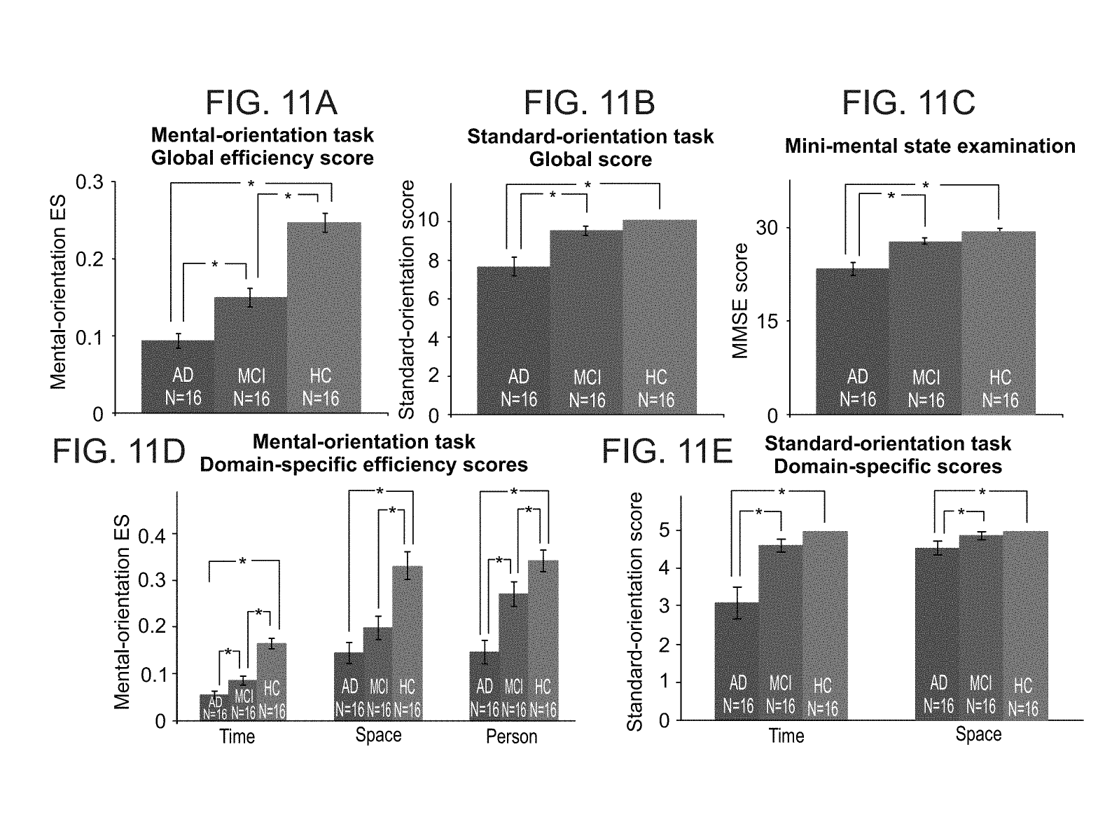

A representative protocol employing the platform, optionally and preferably executes an onboarding, data collection and validation process as illustrated in the flowchart diagram of FIG. 9. The protocol optionally and preferably verifies that the patient is capable of taking the test in order to rule delirium or other physical ailments which impede cognition. The subject-specific task is presented to the subject and the responses are entered. Then, executive functions (e.g., digitalized Trails A&B) are checked for better specificity (e.g., ruling out VD). This is adopted to enable better scoring of the strategy, velocity, and reaction time success rate. Different versions of trails eliminate the learning effect and enable different difficulties for different patients. This enables a short practice test and test. Typically, a repeat task instructions button is employed on the user interface to enable the assessment to include successful or failed execution of a task in short term. Analysis the subject's self correction on a touch screen offers further insight to the status of impairment on the AD/dementia spectrum

The representative protocol may include one or more additional operations. The subject is requested to generate words for a certain letter, and a standard sum of words for each letter is established. A computer assisted device can detect voice-to-text and can count correct and incorrect answers as well as their timing and variability. The representative protocol can also include a computerized version of the symbol digit modalities, executive functions, and shape copying of set of items with different difficulties. The representative protocol can also include a functional abilities questionnaire, and a wellbeing test to rule out depression, anxiety, and aggression. The results of the protocol can be presented in one or more formats including, without limitation, a quick overview, comparison to the norm and comparing to subject's baseline.

### Example 2

### Assessing Individuals Across the Alzheimer's Disease Spectrum

This Example describes a study designed to assess the role of orientation in AD diagnosis, using a subject-specific task. The results were compared to standard orientation and neuropsychological tests. Additionally, the responsiveness of the standard-orientation test to AD-related cognitive decline was examined in a large cohort of patients along the AD spectrum. An fMRI study was conducted in healthy subjects, comparing patterns of activation evoked by subject-specific and standard-orientation tasks to brain regions susceptible to AD pathology.

In this example, the subject-specific cognitive task optionally and preferably is interchangeable referred to as mental-orientation task.

### Methods

### Clinical Study

60 individuals (28 males, mean age: 77.72±7.46, for detailed demographical data see Table 1) participated in the study: 40 patients (20 with AD and 20 with MCI) and 20 age-matched healthy control subjects.

TABLE 1

| Parameters | HC | MCI | AD |
|---|---|---|---|
| Male\|Female | 6\|14 | 10\|10 | 12\|8 |
| Age (years) | 75.3 ± 1.93 | 78.5 ± 1.36 | 79.35 ± 1.6 |
| Education (years) | 15.57 ± 0.81 | 14.15 ± 0.78 | 11.1 ± 0.83 |
| MMSE | 29.4 ± 0.19 | 27.85 ± 0.37 | 22.6 ± 0.74 |
| ACE | 95.6 ± 1.09 | 83.75 ± 2.47 | 59.95 ± 4.46 |
| HIS | 1.75 ± 0.29 | 2.7 ± 0.37 | 3.3 ± 0.37 |

Participants underwent a full neurological examination as well as neuropsychological evaluation that included the Addenbrooke's Cognitive Examination and the Frontal Assessment Battery. Patients from the MCI group were also assessed using the Clinical Dementia Rating (CDR). Patients were recruited from the memory disorders clinic in Hadassah Medical Center and met the National Institute on Aging and the Alzheimer's Association clinical criteria for AD and MCI. All participants provided written informed consent, and the study was approved by the ethics committee of the Hadassah Hebrew University Medical Center.

In the subject-specific task, participants were presented with pairs of stimuli consisting of names of cities (space), events (time), or people (person) (Table 2), and were asked to determine which of the two is closer to them: spatially closer to their current location (for space stimuli), temporally closer to the current time (for time stimuli), or personally closer to themselves (for person stimuli).

Space stimuli consisted of names of cities, distanced 8-150 km from subjects' location. Time stimuli consisted of two-word descriptions of common past events from personal life (e.g., first grandchild) or non-personal world events (e.g., Obama's election). Person stimuli consisted of names of people, familiar to the subject, either acquaintances (family members, friends) or publicly-known people. Prior to testing, subjects reviewed the stimuli, and indicated geographical location and nearby landmarks for space stimuli, approximate year and nearby events for time stimuli, and affiliation for person stimuli. Stimuli which elucidated incorrect answers were removed from further testing. Stimuli in each domain were assigned to one of three distance categories relative to the subjects' own self-location. This procedure yielded an average number of 55±0.93 stimuli (mean±SEM; minimum 45) for all categories per subject.

TABLE 2

| | Distance category | | |
|---|---|---|---|
| Domain | Distance 1 | Distance 2 | Distance 3 |
| Time (years) | 9.29 ± 1.52 | 26.14 ± 1.56 | 47.25 ± 3.69 |
| Space (km) | 15.52 ± 2.48 | 52.30 ± 3.05 | 103.35 ± 10.39 |

11 Pairs of stimuli were generated in each domain (space, time, person), such that the two stimuli never originated from the same distance category. The first pair was excluded from the analysis (learning effect). 5 pairs included stimuli with 1 distance category difference and 5 pairs had a difference of 2. Stimuli were presented in a randomized three-block design, each block dedicated to one domain and containing 11 consecutive trials, with inter stimulus interval of 2000 ms.

Participants were instructed to respond accurately but as fast as possible. Success rates (SRs) and response times (RTs) were recorded. In the standard-orientation test, SRs were recorded for the 10 items included in the MMSE (five regarding the subject's self-location in time and five in space), as well as for the complete MMSE.

In order to control for age and education, Efficiency Scores (ES) were computed by calculating the ratio between the mean SR and RT for each subject and domain separately, for a subset of 48 subjects (16 AD, 16 MCI and 16 HC) that were comparable in age and education (p>0.15, ANOVA and Scheffe's post-hoc tests). A global ES score was calculated by averaging the ES across the three domains. Subsequently, mean ESs were compared across the 3 groups (AD, MCI, HC) using ANOVA and Scheffe's post-hoc tests. Trials with RT displaced by 2.5 standard deviations or more from mean block RT were removed from further analysis. For the MMSE10 SR scores were recorded according to the ACE testing guidelines.

A multivariable ordinal cumulative logistic regression was performed separately for the scores obtained from the subject-specific task and scores obtained from the standard-orientation tasks. In logistic regression, the probability of a binary outcome P(Y=1), here AD and MCI, is estimated using the logit of the sum of multiple independent predictor variables (X₁, X₂ . . . Xₖ), here RTs and SRs, weighted by confidents (α, β₁, β₂ . . . βₖ):

$$P ( Y = 1 \vert {X}_{1} , {X}_{2} … {X}_{k} ) = \frac{1}{1 + {e}^{- ( α + Σ {β}_{1} {X}_{1} + {β}_{2} {X}_{2} … + {β}_{k} {X}_{k} )}}$$

The ordinal cumulative logistic model considers a response variable Y with p categorical outcomes (AD, MCI and HC), denoted j=1, 2 . . . , p, and multiple independent predictor variables (X₁, X₂ . . . Xₖ)—here, SRs for standard-orientation and MMSE, and SRs and RTs for subject-specific task of the present embodiments. In this model, the dependence of Y on X has the following representation:

$$P ( Y \leq {y}_{j} \vert {X}_{1} , {X}_{2} … {X}_{k} ) = \frac{1}{1 + {e}^{- ( {α}_{j} + Σ {β}_{1 j} {X}_{1} + {β}_{2 j} {X}_{2} … + {β}_{kj} {X}_{k} )}}$$

Note that the assumption that the regression coefficient β does not depend on j was relaxed thus allowing examining whether orientation performance in time, space and person contributes differently to the diagnosis of different stages of AD-related decline.

Adhering to the fact that classification is clinically relevant between every two consecutive outcomes (HC-MCI and MCI-AD), six separate logistic regression models were constructed, alternately considering standard-orientation and MMSE SRs and subject-specific SRs and RTs as predictor variables, to estimate the probability of an MCI or AD outcome.

To further determine the diagnostic value of the model produced by the logistic regression, receiver operating characteristic (ROC) curves were plotted. The ROC curve relates proportions of correctly and incorrectly classified predictions over a wide range of threshold levels, with the area under the curve (AUC) accounting for the overall test discriminability. Additionally, an optimal threshold, maximizing sensitivity and specificity, was determined by calculating the Youden's index, and used to determine classification accuracy.

In order to test the observed data set for multicollinearity, variance inflation factor (VIF) was calculated for each of the predictor variables. VIF serves as a measurement of collinearity among the set of predictor variables. Considering a set of k predictor variables (X₁, X₂ . . . Xₖ), VIF for predictor Xⱼ is derived from a linear regression model in which Xⱼ is considered a response variable, and all other predictors as explanatory variables. The regression model produces a coefficient of determination, Rⱼ². VIF for Xⱼ is simply 1/(1−Rⱼ²), and the square root of the VIF (\sqrt{VIF}) is the degree to which the standard error (SEⱼ) has been increased due to multicollinearity.

To control for overfitting of the model to the data, a leave-1-out cross-validation test was conducted. Finally, to further support the classification results, a permutation test, in which outcome labels were randomly shuffled, was performed 1000 times. The aforementioned classification procedure was repeated for each permutation, resulting in a normal distribution of 1000 AUC values. A t-test was performed to determine the probability that the AUC values produced from the unperturbed data belong to the shuffled-AUC distribution.

### Neuroimaging

Given the existing knowledge concerning brain regions affected in AD, characteristic patterns of activations for subject-specific and standard-orientation tasks were established, under the hypothesis that the former will show significant overlap with AD-susceptible regions. To best capture activations, nine healthy participants performing adapted versions of both tasks were recorded, as well as a lexical control task, while undergoing fMRI. The subject-specific task was performed as detailed above. In the fMRI-adapted standard-orientation task participants were presented with stimuli from sets overlapping the subject-specific task sets, and were required to determine which of the two stimuli is indicative of their current location in space, the present time, and personal status. In a lexical control task, participants were presented with stimuli pairs from the same sets but were instructed to indicate which of the words contains the letter “A”.

To assess the selective activations elicited by different experimental tasks, a group-level random-effects general linear model (GLM) analysis was applied. In order to identify the full extent of activation for each domain, domain-specific activations were contrasted separately for the subject-specific task and standard-orientation task with the lexical control task. To directly compare brain regions recruited during each of the two tasks, subject-specific and standard-orientation evoked activations were contrasted with each other across all domains. Subject-specific, standard-orientation and lexical control activity (above rest) were compared across the entire brain by quantifying the number of suprathreshold voxels active for the space, time and person conditions. These were further compared in brain regions susceptible to early AD-related atrophy, including entorhinal, parahippocampal, superior-temporal and temporal pole cortices as well as the amygdala and hippocampus. These regions were grouped to form a single volume of interest (VOI) using the spatial coordinated provided by the AAL atlas. Concordantly, subjects' functional data was normalized into MNI space and subjected to the previously described preprocessing and random-effects group analysis (P<0.05, FDR-corrected, cluster-extent based thresholding corrected). To evaluate and compare mental-orientation's, MMSE10's and lexical control's recruitment of AD-susceptible regions, the number of voxels active for each condition (above rest) and belonged to the AD-susceptible VOI, were quantified.

### Results

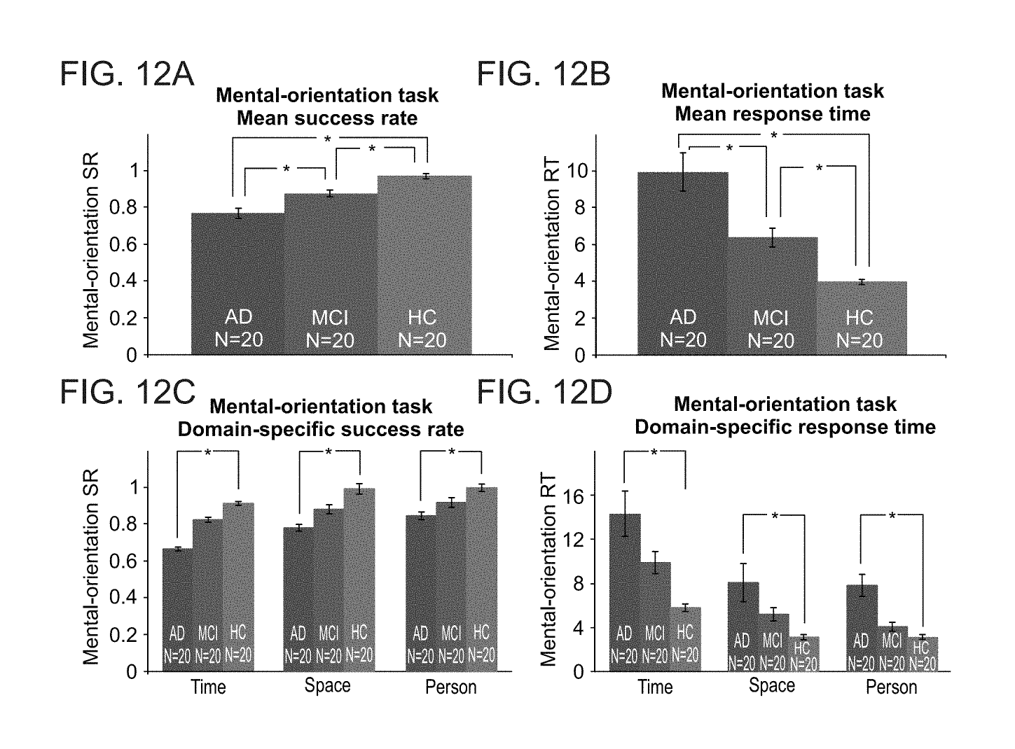

FIGS. 10A-E show behavioral results. Mental-orientation ES showed significant differences between all 3 clinical groups (p<0.05, Scheffe's post-hoc test). Patients with AD scored significantly lower than patients with MCI, and the latter—lower than HCs (mean±SEM: 0.094±0.008[sec⁻¹], 0.158±0.011[sec⁻¹], 0.252±0.013 [sec⁻¹], respectively; FIG. 10A). With respect to the standard-orientation and MMSE scores, patients with AD scored significantly lower (7.07±0.44 and 22.60±0.74, respectively) than patients with MCI (9.60±0.15, 27.85±0.37, p's<0.05, FIG. 10B-C). However, the latter showed comparable results to these of HC (10±0, p=0.54; 29.40±0.19, p=0.08; FIGS. 10B-C).

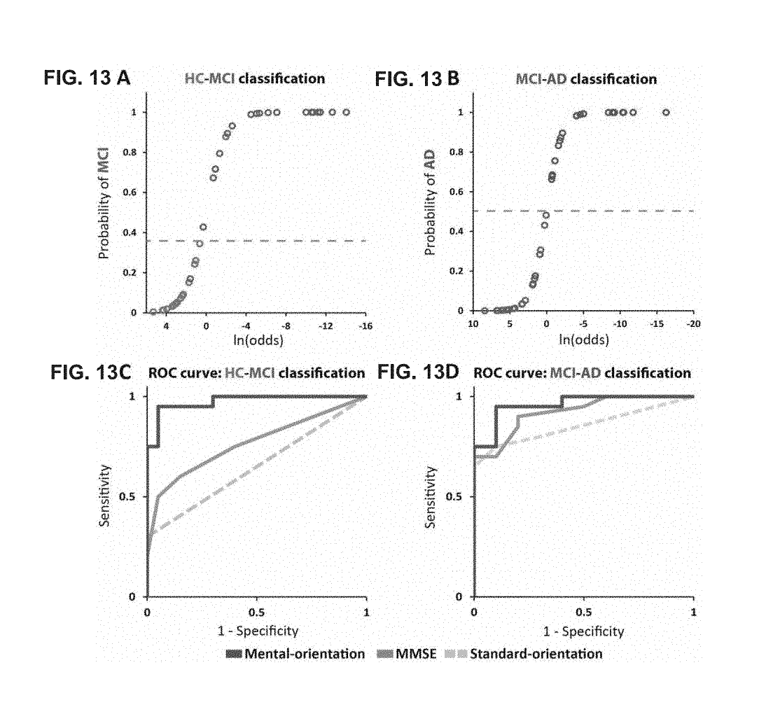

FIGS. 11A-E show age and education comparable subsets. Mean global and domain-specific mental-orientation, standard-orientation and MMSE scores were compared between patients with AD, MCI and HC subjects, comparable in age and education. Efficiency scores for all mental-orientation domains (FIG. 11A) as well as the different domains of time and person (FIG. 11D) showed significant differences between the three clinical groups, while mental-orientation is space was significantly different for HC and patients (ANOVA and Scheffe's post-hoc test, p<0.05). Standard-orientation and MMSE scores were significantly different only between AD and non-AD groups for all domains (FIGS. 11B and 11C) as well as in the time and space standard-orientation sub-scores separately (FIG. 11E).

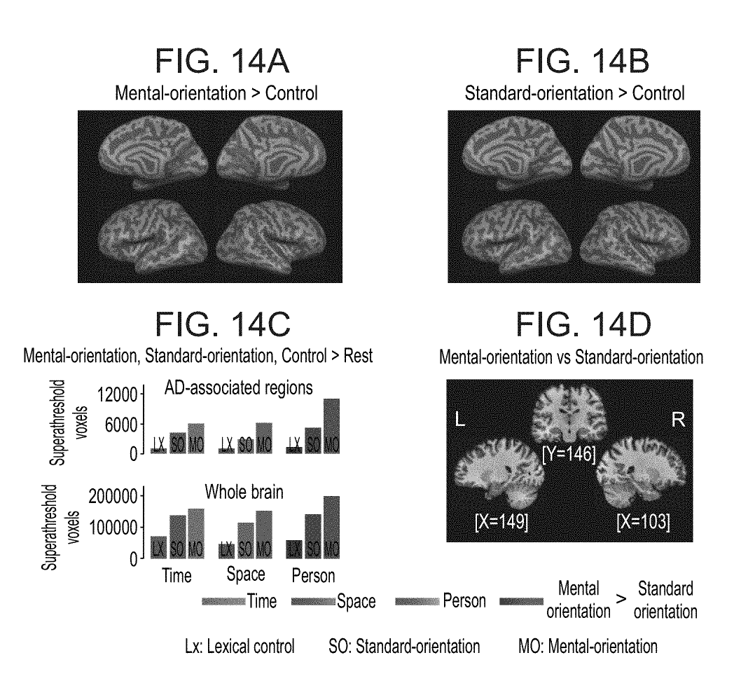

FIGS. 12A-D show SR and RT analyses. Mean global and domain-specific SR and RT were calculated for the mental-orientation task and compared between AD, MCI and HC clinical groups. Significant statistical differences were determined using ANOVA and Scheffe's post-hoc test (p<0.05): FIG. 12A shows combined mental-orientation SRs; FIG. 12B shows combined mental-orientation RTs; FIG. 12C shows Mental-orientation SRs for Space, Time and Person, and FIG. 12D shows mental-orientation RTs for Space, Time and Person. Mean global mental-orientation SR and RT scores produced statistically significant differences between all clinical groups, while domain specific scores produced significant differences mainly between AD and HC.

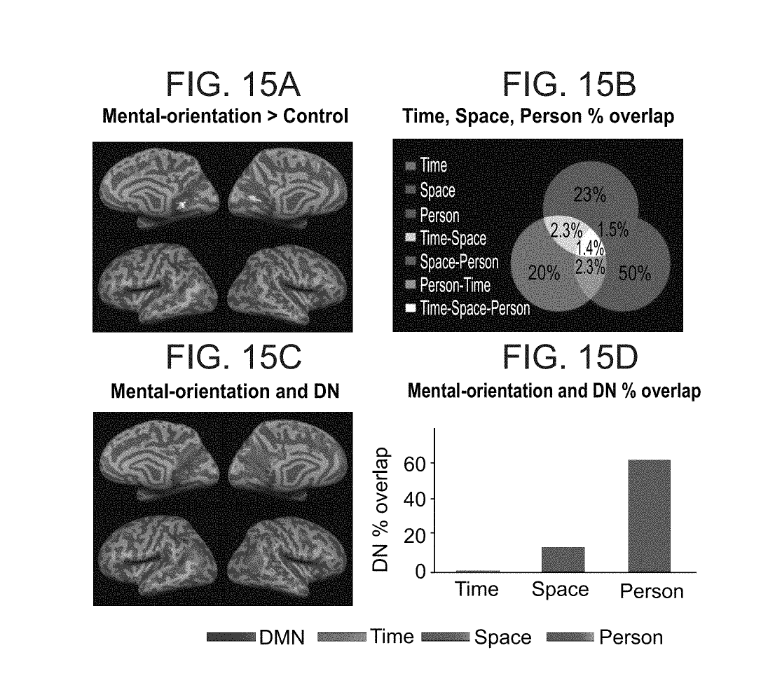

FIGS. 13A-D show machine-learning based analyses. FIGS. 13A and 13B show logistic regression for HC-MCI distinction (FIG. 13A) and MCI-AD distinction (FIG. 13B). FIGS. 13C-D show ROC curves for HC-MCI distinction (FIG. 13C) and MCI-AD distinction (FIG. 13D). The subject-specific task was significantly superior to standard-orientation and MMSE, performing the HC-MCI distinction at 95% accuracy (AUC=0.98, FIGS. 13A and 13C), and the MCI-AD distinction at 92.5% accuracy (AUC=0.94 FIGS. 13B and 13D). MMSE and standard-orientation both produced 50% accuracy for the HC-MCI distinction (AUC=0.77, 0.65, respectively, FIG. 13C), and 85% and 82.5% accuracy for the MCI-AD distinction (AUC=0.92, 0.86 respectively, FIG. 13D).

Concerning the subject-specific task, variance-inflation-factor values were within acceptable range for all variables (VIF<5). Permutation tests showed that the classifications based on the subject-specific task are not compatible with random classification of AUCs (HC-MCI: p<0.0001, MCI-AD: p<0.0004). Leave-1-out analysis revealed 86.25% success of classification.

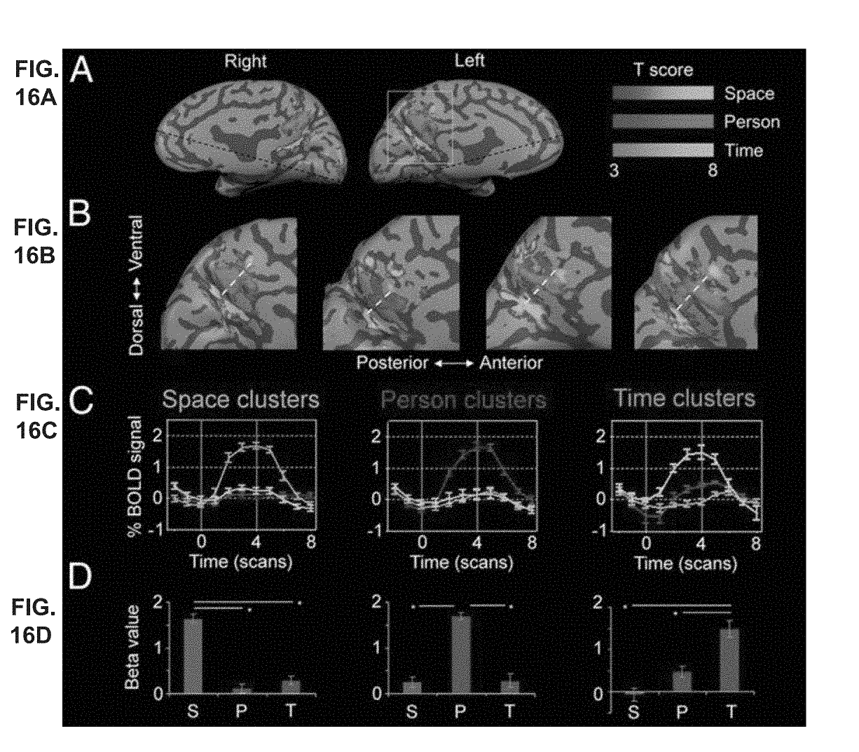

FIGS. 14A-D show evoked brain activity. Under fMRI mental-orientation was shown to activate the precuneus, parietooccipital sulcus, anterior and posterior cingulate cortices, parahipocampal and supramarginal gyri bilateraly, and the left superior frontal gyms, partially overlapping the DN (FIG. 14A). In comparison, standard-orientation activated considerably fewer regions, all locolized to the superior temporal and supramarginal gyri (FIG. 14B). Direct contrast of mental-orientation and standard-orientation activations revealed the subject-specific task to preferentially activate a set of brain regions including the posterior parietal cortex, parieto-occipital sulcus and hippocampus bilaterally. The reverse contrast did not yield any significant activation (FIG. 14C). Quantification of suprathreshold voxels (above rest) revealed significantly increased activation evoked by the mental-orientation over standard-orientation and the lexical control in both whole-brain (499092, 386382 and 170751 voxels, respectively; p<0.0004), and AD-susceptible regions (23103, 12313 and 3371 voxels, respectively; p<0.0004; FIG. 14D).

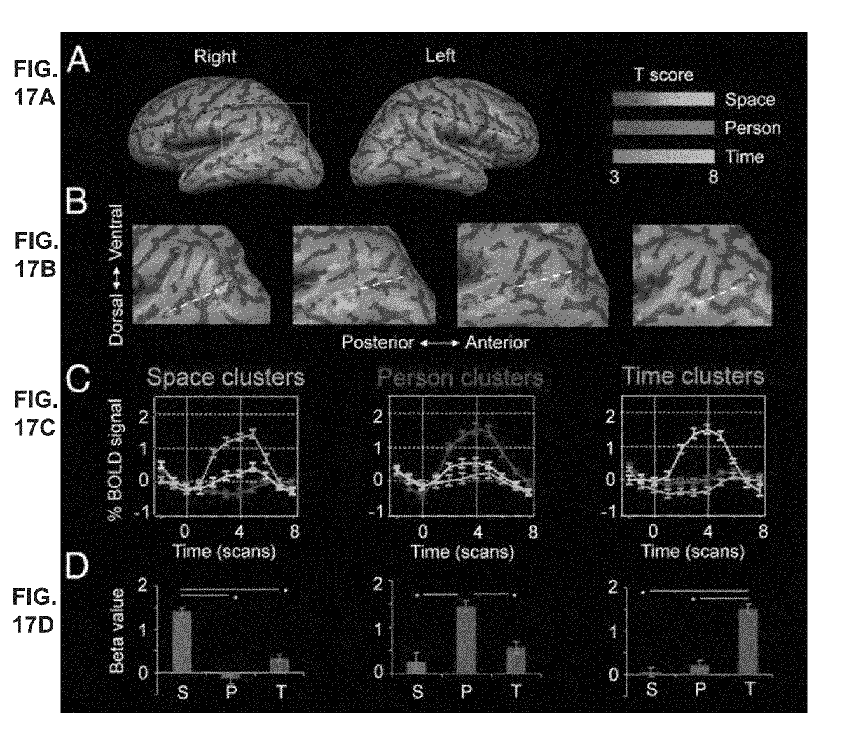

FIGS. 15A-D show Time, Space, Person and Default Network (DN) overlap. Overlap between mental-orientation domains and the DN (FIG. 15A) Overlap between activations in the space, time, and person domains (each contrasted to the lexical control task, p<0.0004, cluster-extent based thresholding corrected). FIG. 15B is a Venn diagram of the percent of overlap between active voxels in each orientation domain, showing a partial overlap between domains. FIG. 15C shows overlay of mental-orientation activations and group DN pattern of activity (including voxels active in individual DN maps in 4 or more of the subjects). FIG. 15D shows the percent of DN activity overlapping with mental-orientation in the different domains: 62% overlap with person, 12% overlap with space, and 0.1% overlap with time.

The present Example demonstrates that the subject-specific task of the present embodiments discriminates between AD, MCI and HC patients on both the group and single-subject levels, unlike standard-orientation or MMSE. Independently, analyzing standard-orientation and MMSE dynamics in a group of longitudinally monitored patients revealed these tests to be unresponsive to deterioration from health to MCI. Contrasting the brain activity underlying mental-orientation and standard-orientation performance using fMRI revealed mental-orientation to preferentially recruit brain regions identified as highly susceptible to AD pathology, including the precuneus, posterior cingulate cortex, parieto-occipital sulcus and hippocampus, unlike the standard-orientation task.

### Example 3

### Brain System for Mental Orientation

In this Example, the neurocognitive system underlying orientation in space, time, and person and its relation to the default-mode network (DMN) is investigated. The subject-specific task of the present embodiments was employed with stimuli in the space (places), time (events), and person (people) domains. High-resolution 7-Tesla functional MRI (fMRI) was used in the study. Each subject was analyzed individually in native space and the results were combined to compare activations for the three domains. The results were compared to the DMN as identified in each individual subject by analysis of resting-state fMRI.

### Methods

Sixteen healthy right-handed subjects (eleven males, mean age 23.9±3.9 y) participated in the study. All subjects provided written informed consent, and the study was approved by the ethical committee of the Canton of Vaud, Switzerland.

The same experimental task was used in all three orientation domains. Stimuli consisted of names of cities (space), events (time), or people (person).

Space stimuli consisted of names of cities in Europe, distanced 50-1,500 km from the experimental location (Lausanne, Switzerland). Time stimuli consisted of two-word descriptions of common events from personal life (e.g., final examinations) or nonpersonal world events (e.g., Obama's election), as well as potential future events of both types (e.g., first child, Mars landing). Person stimuli consisted of names of people, personally familiar to the subject (family members, friends) or famous people (e.g., Barack Obama, Julia Roberts).

Several days before the experiment, participants received a questionnaire and were asked to estimate their spatial distance from each location, temporal distance from each event, and personal distance from each person, on a scale of one to seven, giving rise to seven distance categories. Stimuli were selected from the original questionnaire to obtain five stimuli from each of the seven categories (35 stimuli in total for each domain). To avoid memorization of stimuli, 210 stimuli were rated and only 105 were selected for use in the experiment. To ascertain the consistency of subjects' distance rating, nine subjects were asked to reevaluate the distances 2-3 weeks after the experiment; no significant differences were found between the two ratings (P>0.44), and the average absolute difference in rating was smaller than 1.

Subjects were presented with two stimuli from the same domain (space, time, or person) and were asked to determine which of the two stimuli is closer to them: spatially closer to their current location (for space stimuli), temporally closer to the current time (for time stimuli), or personally closer to themselves (for person stimuli). Therefore, the task and instructions were similar for each orientation domain (space, time, person). To control for distance and difficulty effects (response-time facilitation for stimuli farther apart from each other), subjects' estimates of stimulus's distances were used to select pairs of stimuli with adjacent distances.

Stimuli pairs were presented in a randomized block design, each block containing four consecutive stimuli pairs of a specific orientation domain and distance. Each pair was presented for 2.5 s, and each block (10 s) was followed by 10 s of fixation. Subjects were instructed to respond accurately but as fast as possible. A 5-min training task containing different stimuli was delivered before the experiment. The experiment comprised five experimental runs, each containing 18 blocks in a randomized order. In addition, subjects performed a lexical control task in a separate run, in which they viewed similar stimuli pairs but were instructed to indicate whether or not any of the words contained the letter “T.” Stimuli were presented using the ExpyVR software. After the experiment, subjects rated each task's difficulty, the strategy used, the emotional valence of each stimulus (from 1 to 10), and whether each event was a future or past event. In the inquiry after the experiment, all participants reported not trying to recall these stimuli ratings during the experiment.

Subjects were scanned in a 7T Magnetom Siemens MRI (Siemens Medical Solutions) at the Center for Biomedical Imaging institute at École Polytechnique Fédérale de Lausanne using a 32-channel coil (Nova Medical) to obtain high-resolution functional scans. Blood oxygenation level-dependent (BOLD) contrast was obtained with a gradient-echo echo-planar imaging sequence [repetition time (TR), 2,500; echo time (TE), 25 ms; flip angle, 75°; field of view, 208 mm; matrix size, 124×124; functional voxel size, 1.7×1.7×1.7 mm; generalized autocalibrating partially parallel acquisition, 2]. The scanned volume included 45 axial slices of 1.7 mm thickness with no gap. The high resolution of the scan did not allow for whole-brain coverage, and therefore the scan was limited in the first 10 subjects to the frontal, parietal, and occipital lobes, excluding the temporal pole, anterior medial and lateral temporal lobe, and the orbitofrontal cortex. In the other 6 subjects, the scan included the temporal, parietal, and occipital lobes but excluded the dorsal prefrontal cortex. BOLD scans consisted of six runs (five orientation runs and a lexical control run), each consisting of 160 TRs. In addition, a resting state scan of 120 TRs with identical parameters was performed. T1-weighted highresolution (1 mm×1 mm×1 mm, 176 slices) anatomical images were also acquired for each subject using the MP2RAGE protocol [TR, 5,500 ms; TE, 2.84 ms; flip angle, 75°; field of view, 256 mm; inversion time 1 (TI1), 750 ms; TI2, 2,350 ms].

fMRI data were analyzed using the BrainVoyager software package (R. Goebel, Brain Innovation, Masstricht, The Netherlands), Neuroelf, and Matlab-based software. Preprocessing of functional scans included 3D motion correction by realignment to the first image in the first run, high-pass filtering (up to two cycles in the task scans and 0.005 Hz in the resting-state scan), exclusion of voxels below intensity values of 100, and coregistration to the anatomical T1 images. Runs with maximal motion above a single voxel size (1.7 mm) in any direction were removed from further analyses. Anatomical brain images were corrected for signal inhomogeneity, skull-stripped, and transformed to anterior commissure-posterior commissure orientation. No spatial smoothing or normalization of the voxels was performed, to preserve the high resolution and specificity of individual-subject activity.

A general linear model (GLM) analysis was applied. Predictors were constructed for all conditions, convoluted with a canonical hemodynamic response function, and themodel was independently fitted to the time course of each voxel. Motion parameters were added to the GLM to remove motionrelated noise. Analyses were performed for each subject separately in native space, in a fixed-effect manner by joining the different experimental runs. Data were further corrected for serial correlations and transformed to units of percent signal change.

To identify activations specific to each orientation domain, a balanced contrast between each specific orientation domain (space, time, person) and the average of the other two domains was used. This contrast identified regions responding specifically to only one orientation domain. Each orientation domain was contrasted with the lexical control task. This second contrast enabled detection of overlap of activations between several domains. To exclude activations which did not rise above baseline, a conjunction analysis was performed for each of these contrasts with an additional contrast between the specific orientation domain and rest (baseline). Activations were classified as belonging to one of four regions: (i) the precuneus region—bordered by the marginal, callosal and parieto-occipital sulci, including the cortex inside these sulci; (ii) the prefrontal lobe—anterior to the precentral sulcus laterally and paracentral sulcus medially; (iii) the inferior parietal lobe—posterior to the postcentral sulcus and lateral to the intraparietal sulcus; and (iv) the lateral temporal lobe—anterior to a line drawn between the posterior end of the lateral sulcus and the preoccipital notch. This grouping in each orientation domain separately was used for the analyses of event-related averaging, activation overlap and adjacency analyses, and beta-value extraction from region-of-interest GLM.

To validate the specificity of the activation clusters at the group level, activation clusters were isolated in each subject using the abovementioned contrasts [P<0.05, false discovery rate (FDR)-corrected], with a minimal threshold of 300 voxels. Clusters were grouped according to their anatomical region (precuneus region, inferior parietal, medial or lateral frontal, lateral temporal). A GLM analysis was run for each subject inside each anatomical region, after correction for serial correlations, normalization to the percent of signal change, and addition of motion parameters to the GLM. To avoid circular-analysis bias, the activation clusters were identified using only four of the five experimental runs, and the remaining (independent) run was used for the GLM computation. ANOVAs with Tukey-Kramer post hoc tests were used to compare the beta values for each domain with the beta values for the other two domains, across all subjects. In addition, event-related responses were averaged for each condition, in each activation cluster (again using four runs for cluster identification and the fifth for response measurement).

The event-related responses were averaged across subjects to obtain a characteristic response. Event-related averages were additionally computed for each DMN node, using data from all experimental runs. Random effects GLM and probabilistic-maps analyses were performed on all subjects after spatial normalization and smoothing, to obtain further group-level results. Subjects' functional data were normalized into Talairach space and smoothed using an 8-mm Gaussian kernel. Random-effects analysis was performed on all 16 subjects using the BrainVoyager software. To observe activations in the temporal and frontal lobes, which were scanned in a partial sample of the subjects, probabilistic-maps analysis was performed on these subjects (10 subjects for frontal lobe, 6 for temporal lobe); individual-subjects maps used for this analysis were FDR-corrected and cluster size-thresholded at 20 voxels.

Overlap of domain-specific activity and the DMN. Independent components analysis (ICA) with 30 eigenvalues was performed on resting-state scans, using a gray-matter mask to reduce noncortical noise. The DMN was identified by searching for a component that included the medial prefrontal, posterior cingulate, and inferior parietal cortices. A component clearly corresponding to the DMN was identified in 13 of the 16 subjects; in the remaining three, no DMN component could be identified, and they were therefore excluded from this analysis. Overall overlap between the DMN and orientation-related regions was computed by counting DMN voxels that were active in a specific domain (identified using a contrast between each orientation domain and the other two domains) and dividing by the total number of DMN voxels. The opposite overlap percentage was computed by counting DMN voxels that showed domain-specific activity (contrast between each orientation domain and the other two domains) and dividing by the sum of all domain-specific active voxels.

Centers of mass were computed for each activation cluster (contrast between each domain and the other two domains) in the precuneus/parietal lobe. Precuneus clusters were rotated by −45° to obtain a rostral-caudal orientation. In each subject where all three clusters (space, time, and person) were identifiable, each cluster's location on the y axis was compared with the other two clusters across subjects using Wilcoxon's signed-rank tests (separately for the precuneus region and parietal lobe).

For each contrast, all of the active voxels were segregated into right and left hemisphere activations using BrainVoyager automatic hemisphere segregation. Voxels were counted in each hemisphere and compared using twotailed paired-sample t test to identify laterality preferences.

To identify overlap between regions, activation clusters were isolated from the contrast between each domain and the control task. Overlap was computed by the percent of voxels significantly active in two or three of the contrasts, compared with the total number of active voxels. Percentages of overlapping voxels were averaged across subjects.

Gaps were computed as the minimal Euclidean distance between the borders of each pair of clusters, in each hemisphere separately. In the case of overlapping activity, overlap extent (maximal Euclidean distance between activation borders inside the overlapping region) was represented by a negative value. Activation clusters were taken from results of the contrast between each domain and the lexical control and the contrast between each domain and the other two domains between each pair of orientation domains in each region. Measuring the effect of emotional valence, distance, and stimulus length. To measure the effect of emotional valence, distance from current location, and stimulus length, the data from the postexperiment questionnaires (averaged across the two simultaneously presented stimuli) was used to create parametrically modulated domain-specific regressors. Specific regressors were separately created for events, indicating whether they happened in the past or will happen in the future. GLM analysis was applied as above with these regressors to evaluate their contribution to the signal. Measuring the effect of response times on brain activations. To measure the effect of response times on the data, a new design matrix was created with the addition of a response-time regressor (z-transformed to orthogonalize it from the existing orientation-domains regressors, and convolved with a hemodynamic response function). A region-of interest GLM was performed with the three orientation domain predictors and the response-time predictor, in each activation cluster identified using the contrasts between orientation domains and other domains, as described in Functional MRI analysis.

### Results

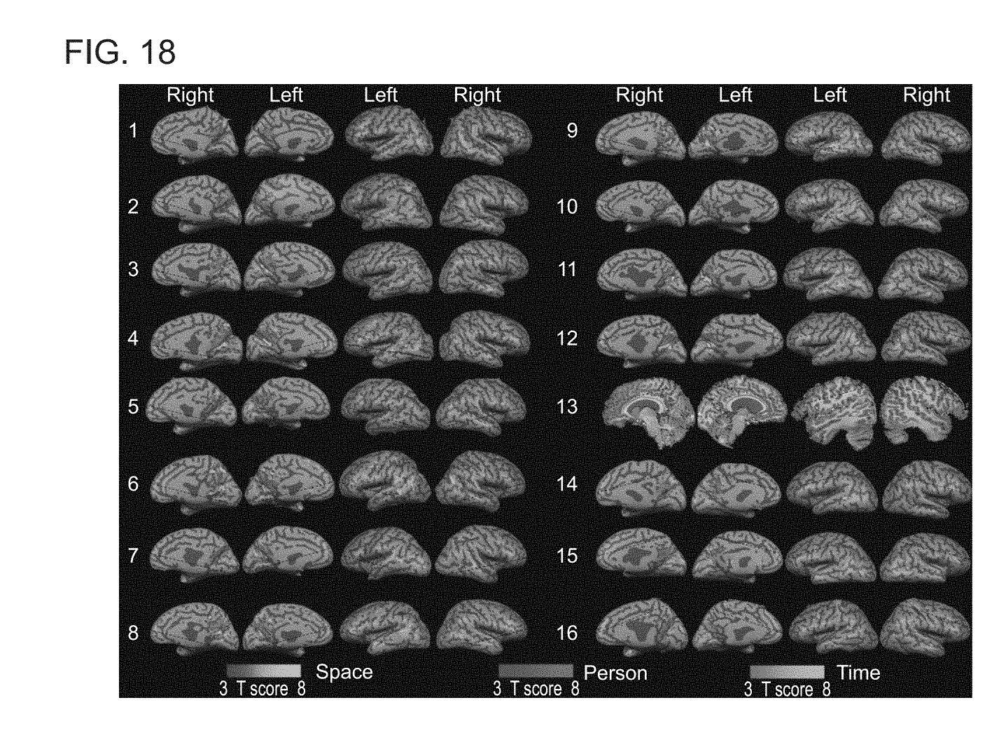

FIGS. 16A-D show midsagittal cortical activity during orientation in space, time, and person. FIG. 16A shows domain-specific activity in a representative subject, identified by contrasting activity between each orientation domain and the other two domains. The precuneus region is active in all three orientation domains, and the medial prefrontal cortex only in person and time orientation (P<0.05, FDR-corrected, cluster size >20 voxels). Dashed black lines represent the limit of the scanned region in this subject. FIG. 16B shows precuneus activity in four subjects, demonstrating a highly consistent posterior-anterior organization (white dashed line); all other subjects showed the same activity pattern. FIG. 16C shows that group average (n=16) of event-related activity in independent experimental runs demonstrates the specificity of each cluster to one orientation domain. Lines represent activity in response to space (blue), time (green), and person (red) conditions. Error bars represent SEM between subjects. FIG. 16D shows group average of beta plots from volume-of-interest GLM analysis, showing highly significant domain-specific activity. Error bars represent SEM between subjects. P, person; S, space; T, time.

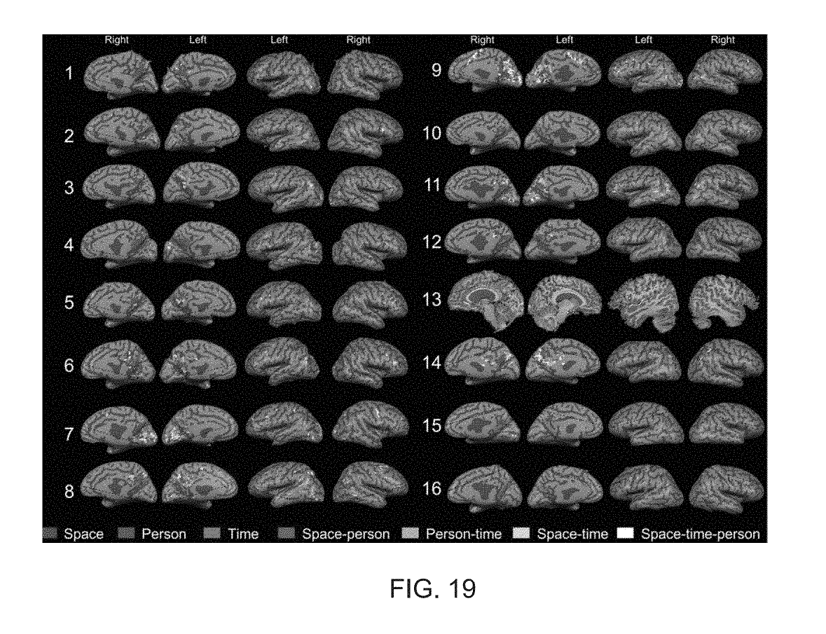

FIGS. 17A-D show lateral cortical activity during orientation in space, time, and person. FIG. 17D shows domain-specific activity in a representative subject, identified by contrasting activity between each orientation domain and the other two domains (P<0.05, FDR-corrected, cluster size >20 voxels). The inferior parietal lobe (IPL) is active in all three orientation domains, and the temporal lobe mostly for time but also for person orientation. Notice the strong left lateralization of time activations. FIG. 17B shows IPL activity in four subjects, demonstrating a consistent posterior-anterior organization (white dashed line): All other subjects showed the same activity pattern. FIG. 17C shows group average (n=16) event-related plots from independent experimental runs. FIG. 17D shows group average of beta plots from volume-of-interest GLM analysis. Colors and symbols are as in FIGS. 16A-D.

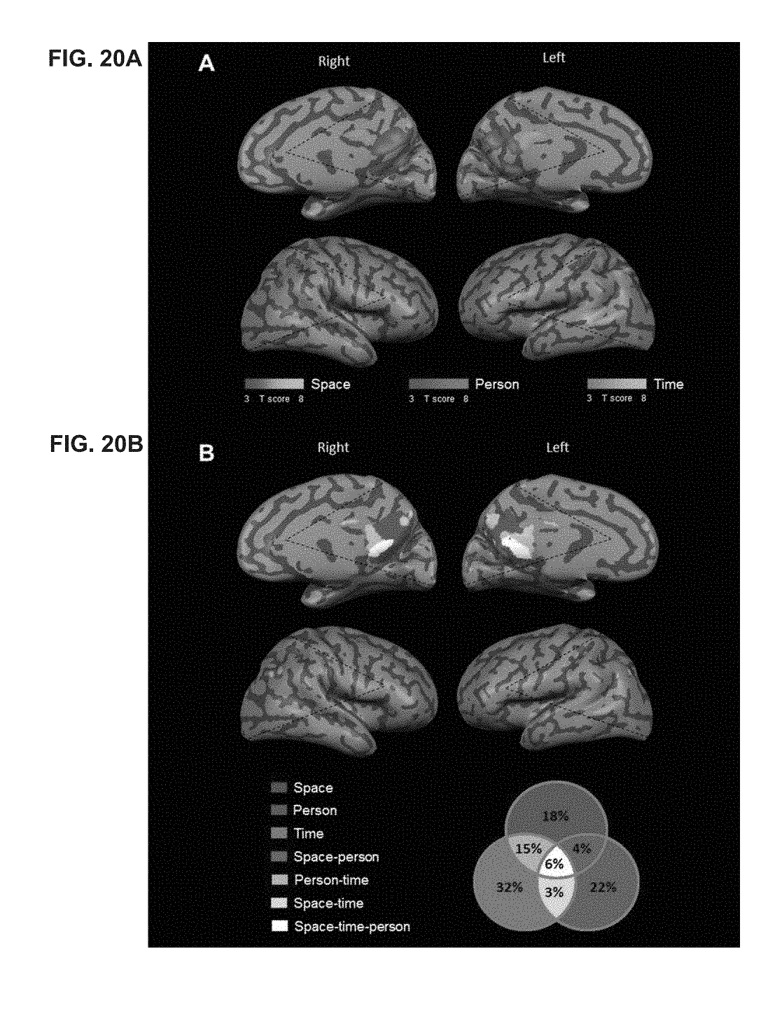

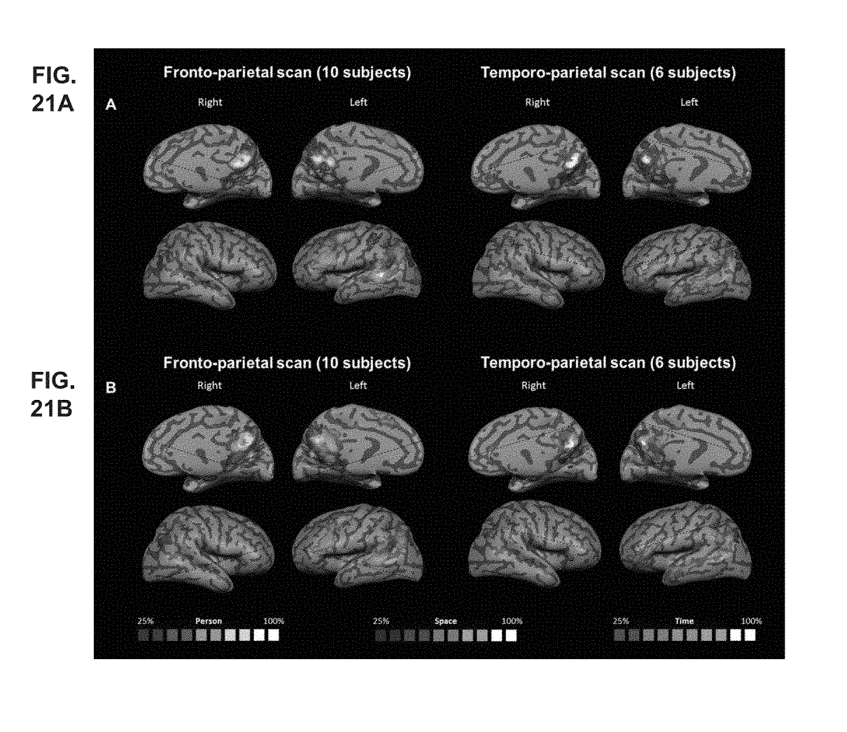

FIG. 18 shows cortical activity during orientation in space, time, and person in individual subjects. Domain-specific activity is shown for all 16 subjects, obtained by contrasting activity between each orientation domain and the other two domains (P<0.05, FDR-corrected, cluster size >20 voxels). Dashed lines represent the limit of the scanned region in each subject. Subject 13 could not be transformed to an inflated brain representation due to technical reasons and is therefore presented by representative slices. Notice the consistent pattern of activity in the inferior parietal, medial parietal, frontal and temporal cortices FIG. 19 shows overlap between activations in the different orientation domains in individual subjects. Overlapping and nonoverlapping activity is shown for all 16 subjects, obtained by contrasting activity between each orientation domain (space, time, and person) and a lexical control task (P<0.05, FDR-corrected, cluster size >20 voxels). Significant overlap was found in 14/16 subjects. Subject 13 could not be transformed to an inflated brain representation due to technical reasons.

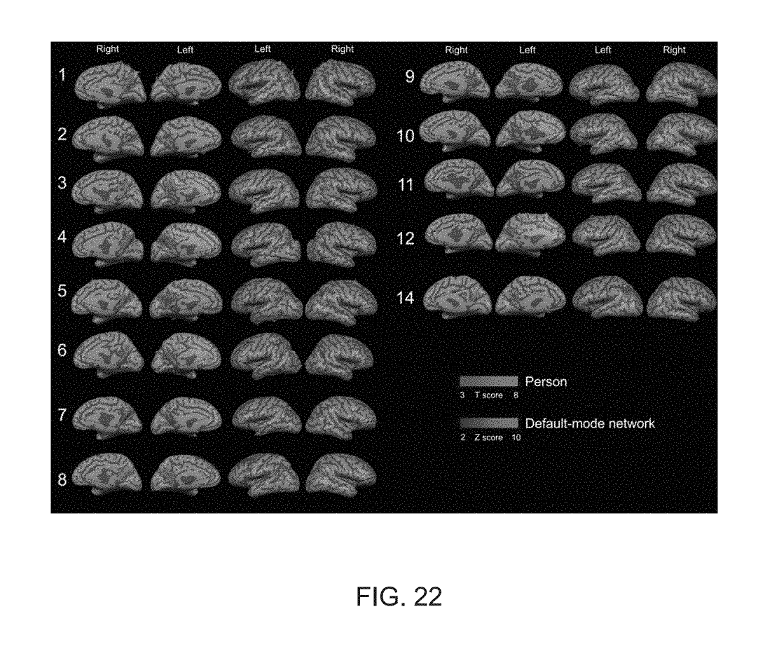

FIGS. 20A-B show random-effects group analysis. All 16 subjects were analyzed with a random-effects group analysis. FIG. 20A shows contrast between each orientation domain (space, time, or person) and the other two domains, indicating regions of domain-specific activity (P<0.05, FDR-corrected, cluster size >20 voxels). FIG. 20B shows contrast between each orientation domain and the lexical control task (P<0.05, FDR-corrected, cluster size >20 voxels). Dashed lines indicate borders of regions scanned in all 16 subjects, on which the analysis was performed. The Venn diagram (bottom right) demonstrates the prominent overlap between activations in the precuneus and inferior parietal regions.

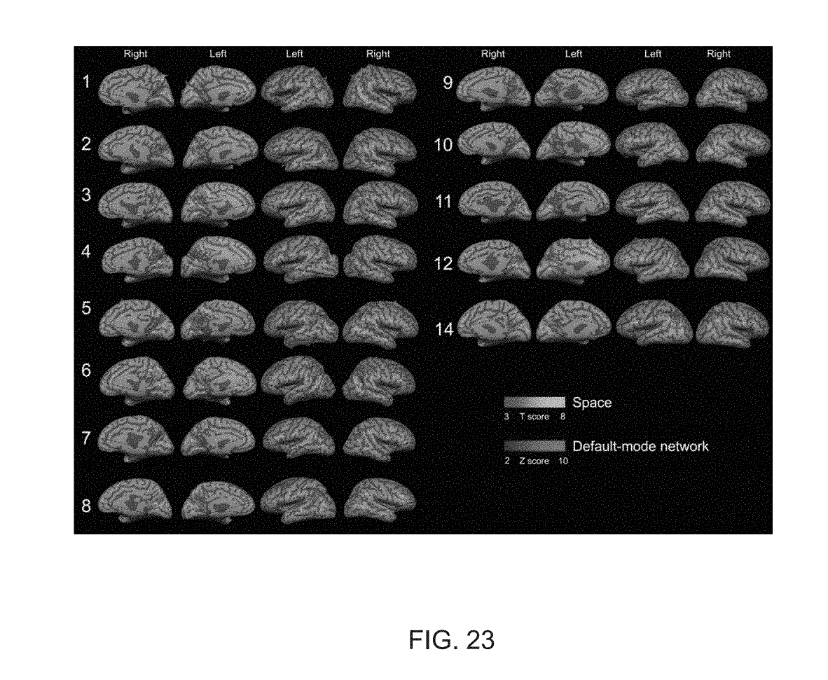

FIGS. 21A-B show probabilistic-maps group analysis. Two groups of subjects were analyzed separately based on the coverage of their functional scans: 10 subjects scanned with frontal and parietal coverage (Left), and 6 subjects scanned with temporal and parietal coverage (Right). FIG. 21A shows contrast between each orientation domain (space, time, or person) and the other two domains, indicating regions of domain-specific activity. FIG. 21B shows Contrast between each orientation domain and the lexical control task. The probabilistic maps are thresholded at 25% of subjects of each group.

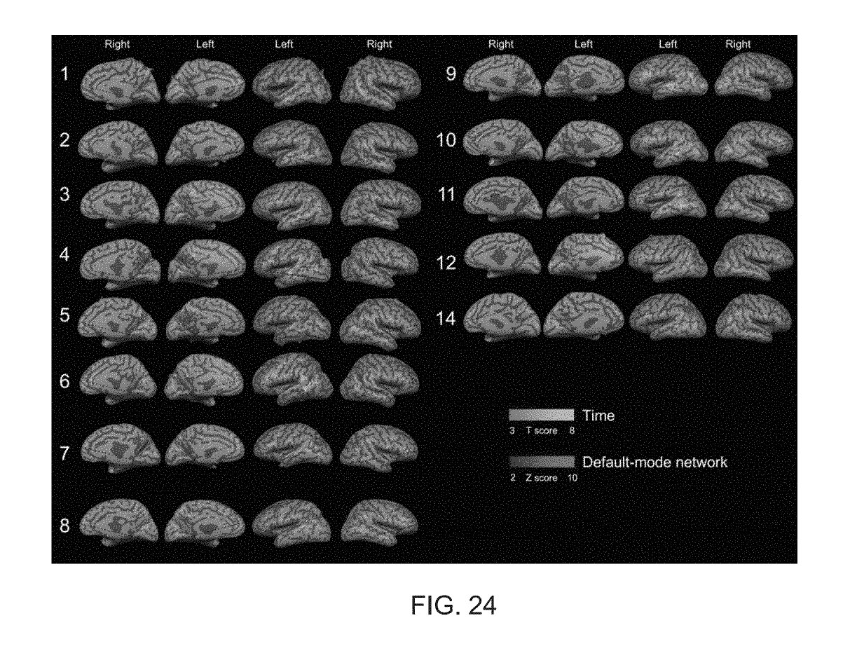

FIG. 22 shows overlap between the default-mode network (DMN) and activity during orientation in the person domain for individual subjects. An ICA component clearly corresponding to the DMN could be identified in 13 out of the 16 subjects. A clear overlap is apparent between the DMN and regions of person orientation.

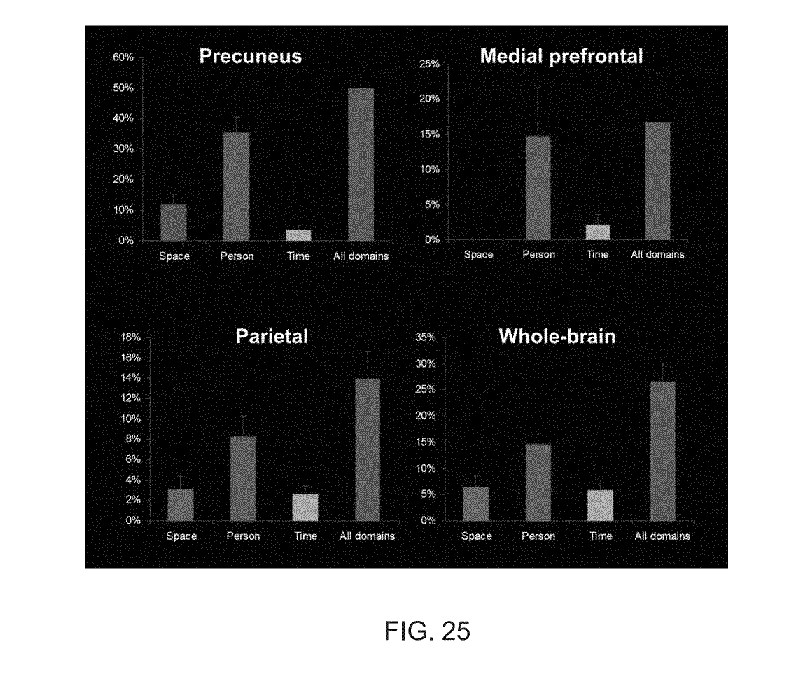

FIG. 23 shows overlap between the default-mode network (DMN) and activity during orientation in the space domain for individual subjects. Regions of spatial orientation generally lie outside and adjacent to the default-mode network although some overlap exists.

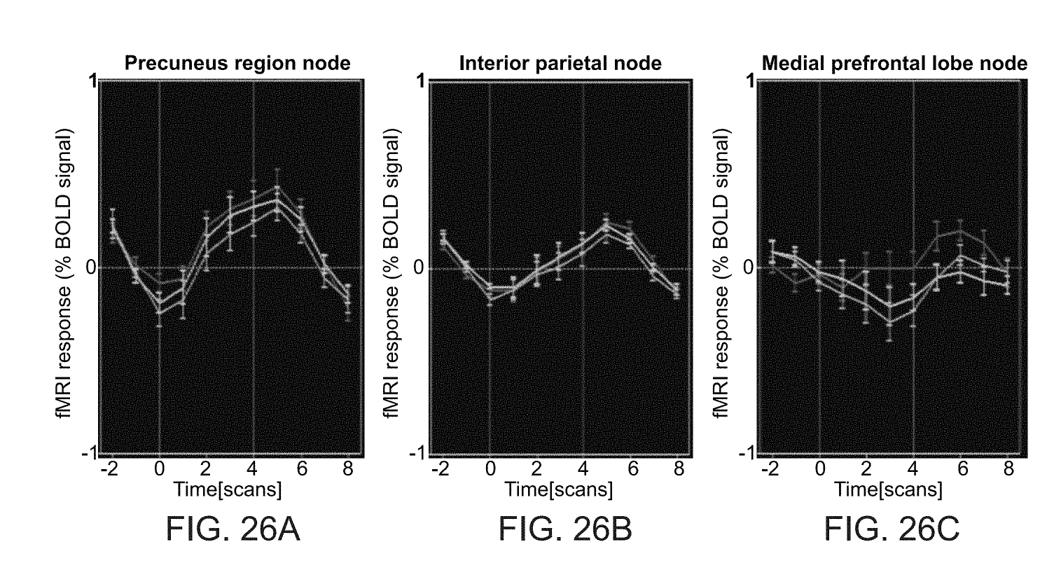

FIG. 24 shows, overlap between the default-mode network (DMN) and activity during orientation in the time domain for individual subjects. Regions of temporal orientation generally lie outside and adjacent to the default-mode network, although some overlap exists. Notice also the strong left-lateralization of time activations.

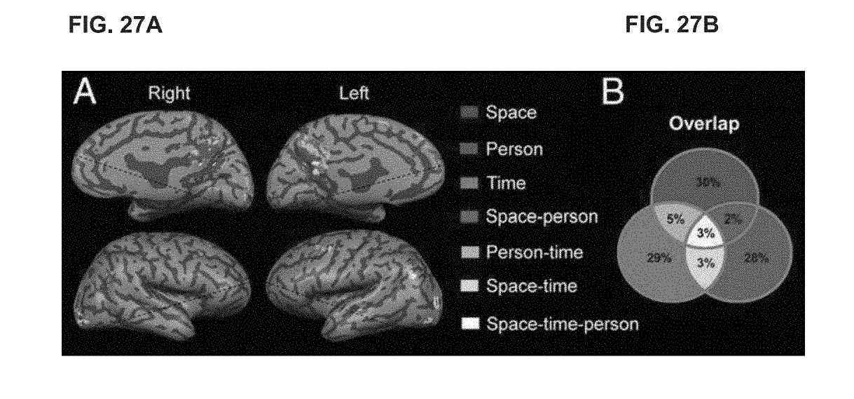

FIG. 25 shows average DMN overlap with orientation domains for individual subjects. In each brain region, the average overlap of DMN with each domain-specific region (contrast between each orientation domain and the other two domains) is calculated as the number of DMN voxels in each domain divided by the total number of DMN voxels.

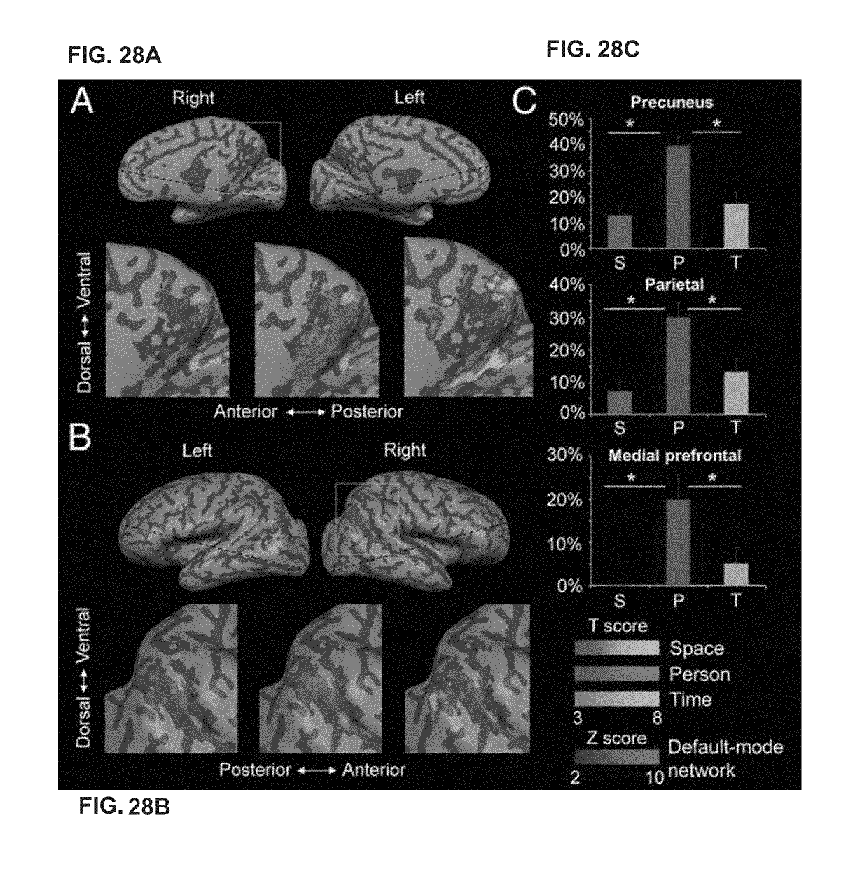

FIGS. 26A-C show event-related time courses from default-mode networks nodes, for the different orientation domains. The default-mode network is similarly active across all orientation domains in the precuneus and inferior parietal lobes, and only for the person domain in the medial prefrontal lobe (blue, space; red, person; green, time; error bars represent SEM between subjects).

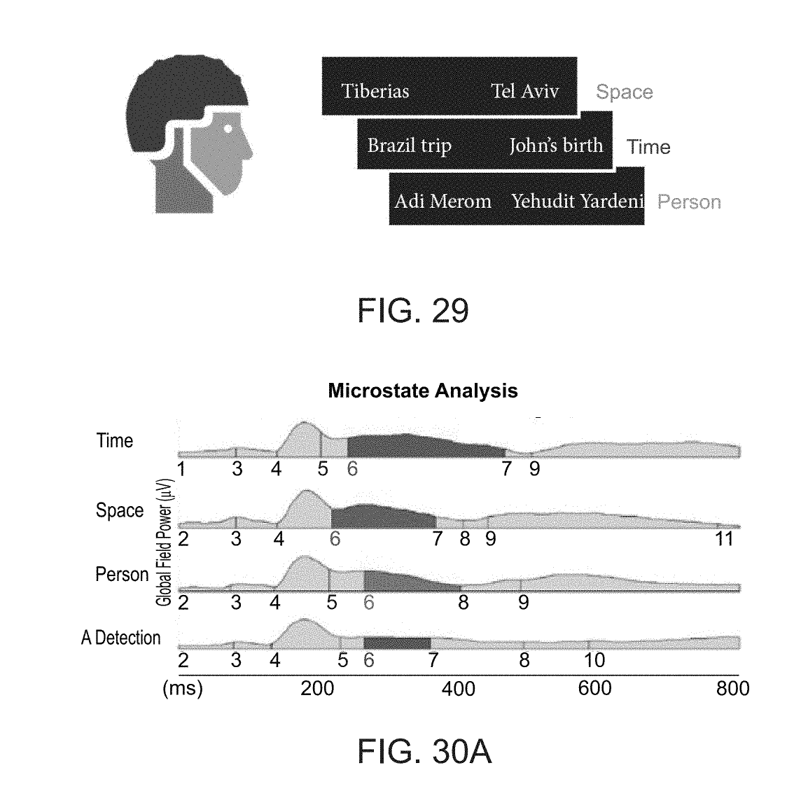

FIGS. 27A-B show overlap between activations in the space, time, and person domains. FIG. 27A shows overall orientation-related activity in a representative subject, identified by contrasting activity between each orientation domain and the lexical control task, showing overlap between regions (P<0.05, FDR-corrected, cluster size >20 voxels). FIG. 27B shows group average of the percent of overlap between active voxels in each orientation domain, demonstrating a partial overlap between domains.

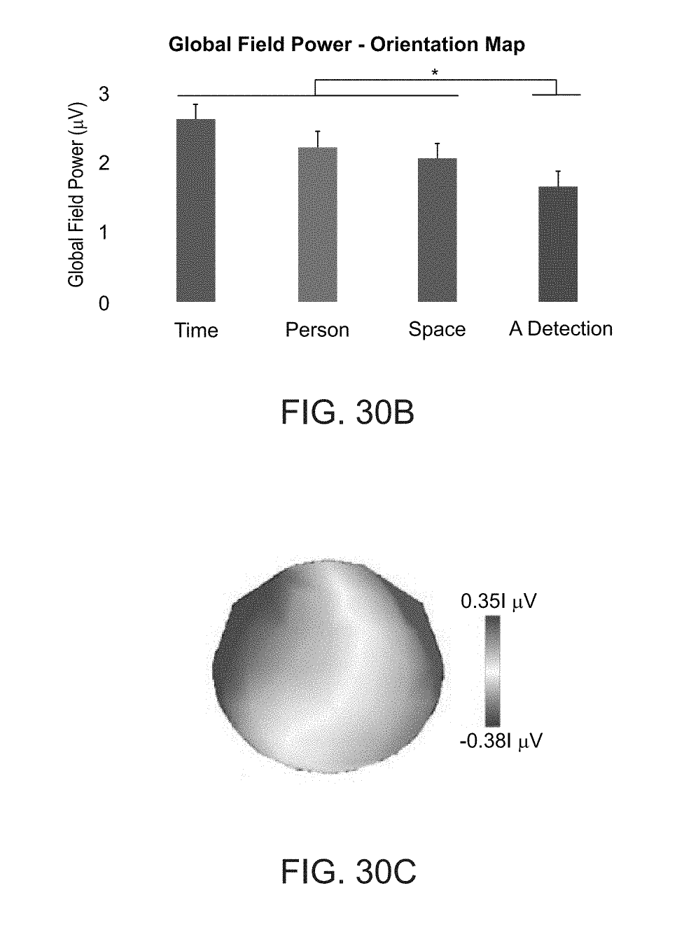

FIGS. 28A-C show overlap of orientation activity with the default mode network (DMN). The DMN was identified using resting-state fMRI in each individual subject. The DMN is presented for a representative subject, overlaid with activity during the orientation task in space, time, and person (identified by contrasting activity between each orientation domain and the other two domains). (A) Midsagittal view, focus on the precuneus. (B) Lateral view, focus on the IPL. (C) Average percent, across subjects, of DMN voxels from all voxels active specifically for a single orientation domain. DMN voxels were found most prominently in the person domain (two-tailed t test, all P<0.01) although some were found also in the time and space domains. P, person; S, space; T, time.

fMRI analysis for each domain of orientation (space, time, and person) revealed an identical pattern of brain activation for all subjects: for all three domains, activations were found in the precuneus and the adjacent posteriorcingulate cortex, regions within the IPL, and parts of the superior frontal sulcus and occipital lobe. In the time and person domains, activation was additionally found at the mPFC and the superior temporal sulcus.

Analysis of activations for the three domains revealed orientation-related regions, which are consistently organized in each individual subject. In all subjects, the same pattern of a posterior-anterior axis of activation was found for space, person, and time, respectively. In the precuneus region, space orientation activated a posterior region around the parieto-occipital sulcus, person orientation activated the precuneus and posteriorcingulate cortex, and time orientation activated the anterior precuneus (P<0.05, Wilcoxon signed-rank test). The IPL showed an identical order of posterior-anterior activation: Space orientation activated a posterior region near the intraparietal sulcus, person orientation activated posterior parts of the angular gyrus, and time orientation activated the anterior angular gyrus, extending into the temporal lobe (P<0.05, Wilcoxon signed-rank test). In the mPFC, activity for person orientation was always more anterior than for time orientation (P<0.05, Wilcoxon signed-rank test). Timeorientation activity was found mostly in the left hemisphere (P<0.01, two-tailed paired-samples t test) whereas person and space activations were found bilaterally with no significant hemispheric preference (P=0.41, P=0.26 respectively).

To further validate the specificity of the identified activations and obtain group-level statistics, intraregional general linear model (GLM) and average event-related activity were computed for each region which had an ordered activation pattern (precuneus, IPL, mPFC) and for each orientation domain, and were compared across subjects. These results were computed from a separate experimental run than those used to identify the region of interest, ensuring that the domain-specific activation of each region was independent of its identification. These analyses showed that the domain-specific regions of interest responded consistently and specifically to their preferred orientation domain and not to other domains, across all subjects and regions (all P values <0.001, Tukey-Kramer post hoc test). Random-effects GLM group analysis and a probabilistic-maps group analysis provided results similar to those obtained from single subjects.

The finding of domain-selective regions for orientation revealed a partial anatomical segregation between them. To determine the interrelations between domains, each domain's activity was contrasted with a lexical control task and checked for overlapping activations. At the individual subject level, most voxels (87%) were found to be domain-specific, and 13% of the voxels were activated in response to two or three domains. At the group level, analyses demonstrated overlap of 28% between domains in the precuneus region and IPL. Analysis of the average gap between orientation-related activations revealed no gaps when considering the full extent of orientation-related regions and a gap of 1-7 mm between domain-specific regions in the precuneus and lateral parietal lobe. The results of these overlap and adjacency analyses suggest the existence of core processing for the different orientation domains.

The relation between the DMN and the orientation-related regions was examined. The DMN was identified in a separate resting-state run, using independent-components analysis (ICA) for each individual subject, and was compared with subjects' orientation-related regions. This comparison demonstrated a significant overlap in the precuneus region because 50% of DMN voxels were active during mental orientation (identified using the contrast between each orientation domain and the other two domains). Overlap was also evident in the IPL and mPFC (14% and 17% of voxels, respectively). The relation between the DMN and regions related to each domain (space, time, and person) was also tested. Most of the DMN voxels active during orientation were within personorientation regions (32%), significantly more than in space (12%) and time (10%) regions, across the whole brain (P<0.01, Tukey-Kramer post hoc test). The activity in each DMN node (precuneus, IPL, and mPFC, as identified in the resting-state scan) in response to the orientation task in each domain was also tested. The IPL and precuneus nodes were active for all domains with similar average blood oxygenation level-dependent signal strength, and the mPFC for the person domain.

The functional examination of brain activity during orientation in space, time, and person revealed several findings. Specific regions were found to be active for each orientation domain (space, time, or person) in the precuneus and posterior cingulate cortex, IPL, mPFC, and lateral frontal and lateral temporal cortices. These domain-specific regions are adjacent and partially overlapping and are organized along a posterior-anterior axis. All orientation-related regions have a prominent overlap with the DMN, and DMN nodes responded similarly to the different orientation domains.

The present Example demonstrates that orientation domains have an intrinsic organization in the precuneus region, IPL, and mPFC and support a model of a general orientation system with distinct domain-specific divisions and a common functional core.

### Example 4

Orientation Activation Along the Alzheimer's Disease Spectrum

Disorientation is a hallmark of AD, which manifests in the impaired processing of the relations between the behaving self to space (places), time (events), and person (people). This Example investigates the orientation system under electrical neuroimaging, first in healthy young adults, and subsequently in people along the AD spectrum from health through MCI to AD.

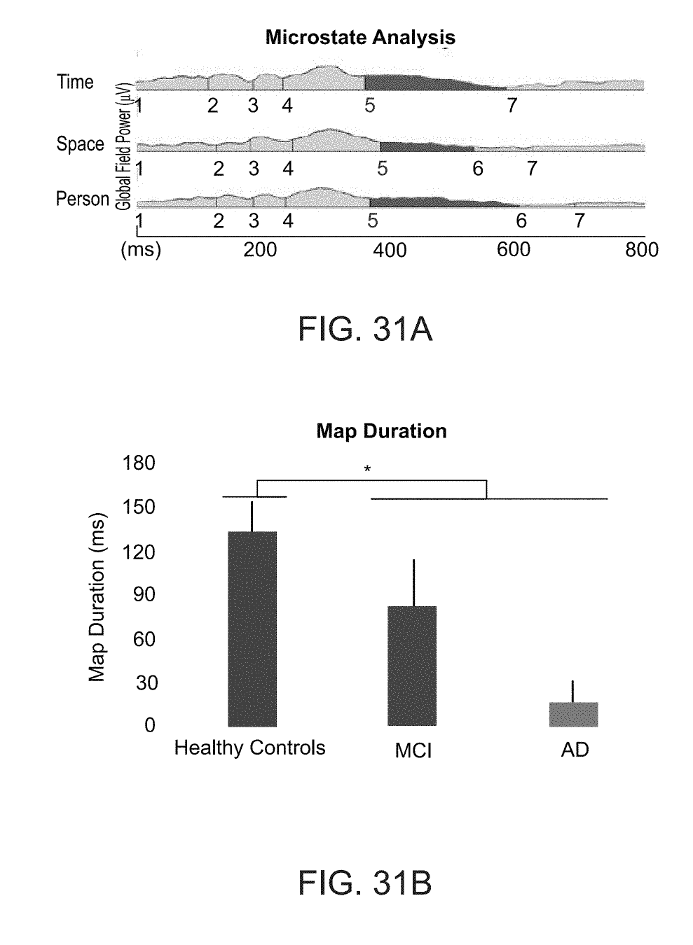

A first experiment included young healthy subjects. Multichannel (64 electrodes) EEG signals were recorded from 18 young healthy subjects, while the subjects performed individually tailored mental-orientation tasks. The subjects were presented with two stimuli from the same orientation domain (Places, Events, People), and were asked to determine which of the two stimuli is closer to them. Representative examples of presented stimuli are illustrated in FIG. 29. In addition, subjects performed a non-orientation lexical control task. In this example the lexical control task included determining which word contains the letter “A”). A second experiment included patients along the AD-spectrum (AD—n=2; MCI—n=2) and healthy age-matched controls (n=7), with the same task and method. The microstate analysis identified a specific EP map representing performance of mental orientation. The EP maps were fitted to the individual subjects in different clinical conditions to enable statistical analysis in the individual subject level. These maps were further localized using linear autoregressive model to identify underlying brain generators.

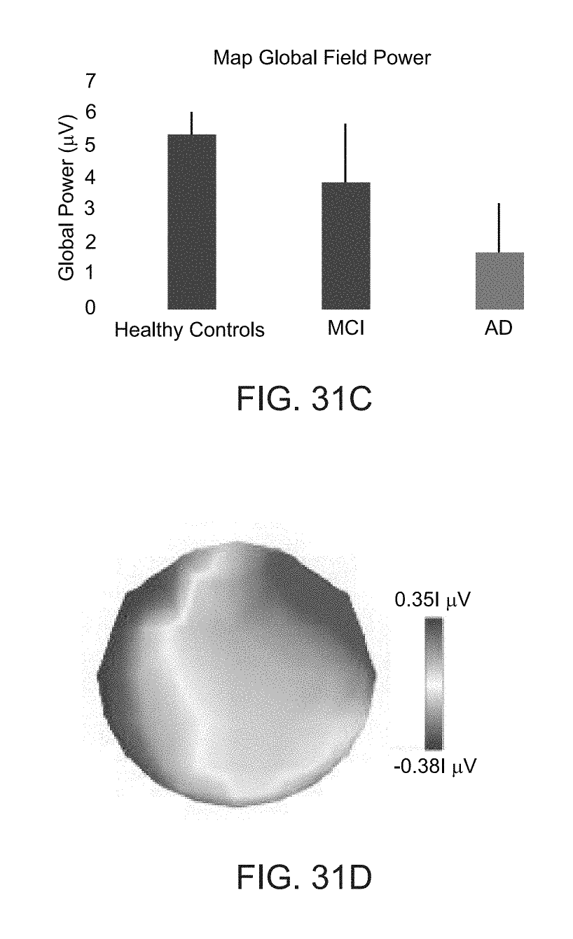

FIGS. 30A-C show the results of microstate analysis applied to the data collected during the first experiment. FIG. 30A shows segments of stable map topography in space, time, person and a control condition under a global field power curve from 0 to 800 ms. An EP map, found at about 280-500 ms (FIG. 30B) was stronger for orientation conditions compared to the control condition (p<0.05). FIG. 30C shows the topography of this EP map.

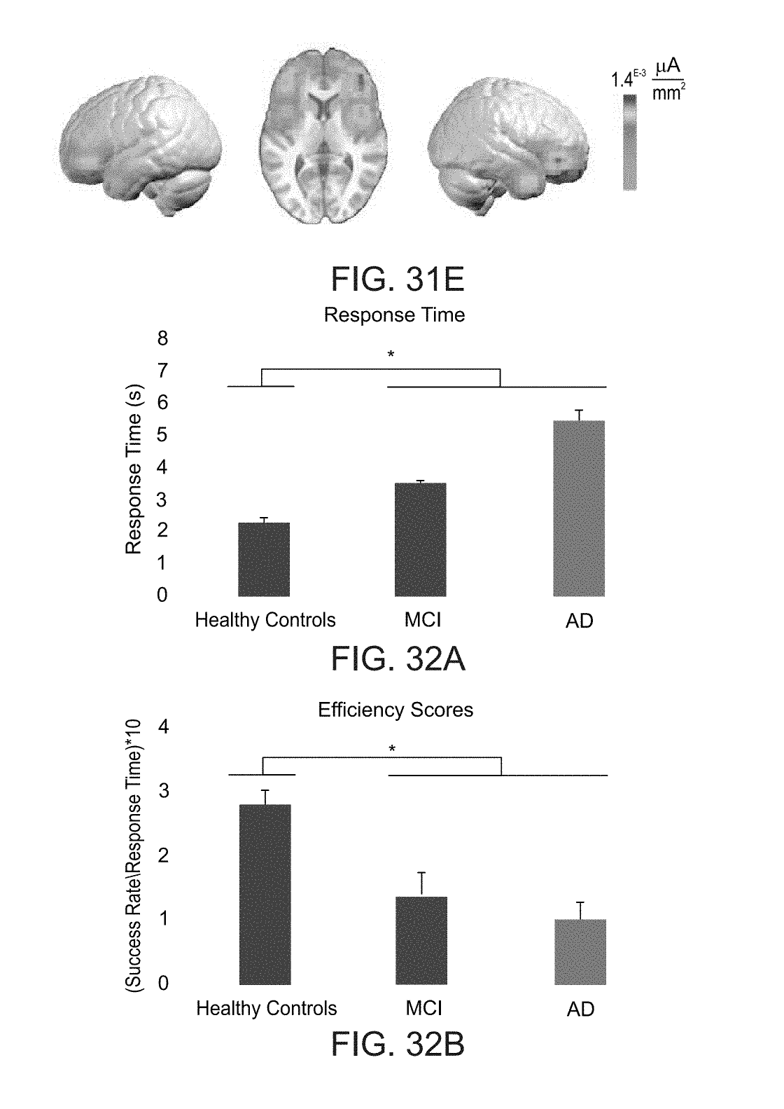

FIGS. 31A-E show results obtained in the second experiment. FIG. 31A shows EP maps in space, time, person under the global field power curve from 0 to 800 ms. An EP map (purple), found at about 360-560 ms, (FIGS. 31B and 31C) was significantly shorter in MCI and AD patients compared to controls (F(28,1)=7.20, p<=0.05). FIG. 31D shows the topography of this EP map, and FIG. 31E shows localization of the mental orientation map bilaterally to the anterior temporal lobe and to the right inferior frontal cortex.

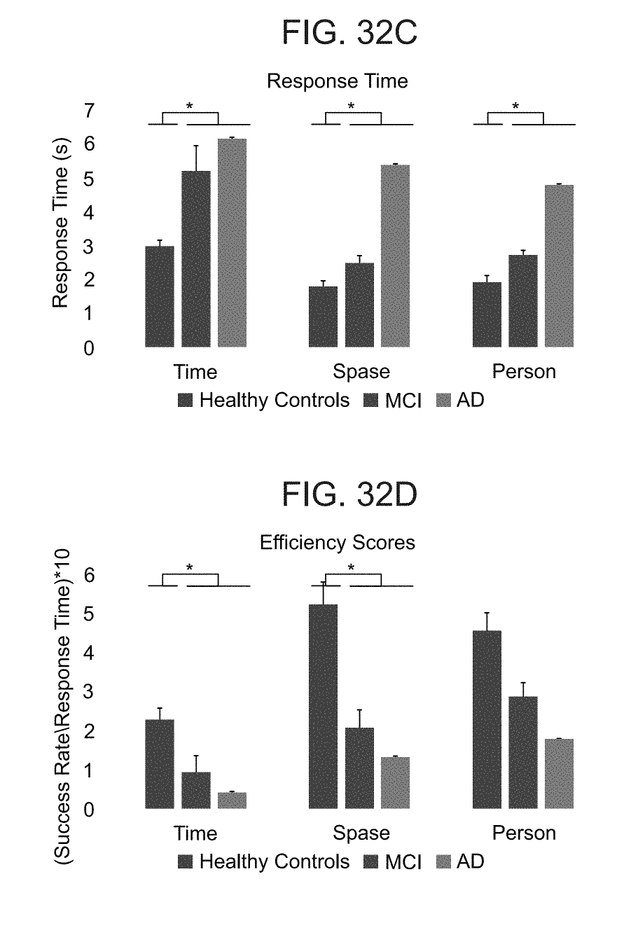

FIGS. 32A-D show mean reaction times and efficiency scores (success rate*10/response time) for the different domains (Time, Space and Person) and clinical conditions. Reaction times were longer and efficiency scores were lower for MCI and AD patients compared to age-matched healthy controls.

In the first experiment, an EP map of mental-orientation that was longer for the time domain than space and person, and almost absent in a lexical-control task was identified (FIGS. 30A-C), corroborating with behavioral results. In the second experiment, patients performance deteriorated along the AD-spectrum as measured by efficiency score (success-rate/response-time; F(28,1)=10.13, p<0.01) (FIGS. 32A-D). A distinct EP map was found at about 360-560 ms which resembled (84.5%) the orientation-map identified in the first experiment (FIGS. 30C and 31D). This orientation map was significantly shorter in MCI and AD patients compared to controls (F(28,1)=7.20, p<=0.05) (FIGS. 31B-C). The orientation map was localized bilaterally to the inferior frontal lobe and to the left medial-temporal lobe (FIG. 31E).

As used herein the term “about” refers to ±10%.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Monacelli A M, Cushman L A, Kavcic V, Duffy C J (2003) Spatial disorientation in Alzheimer's disease: the remembrance of things passed. Neurology 61: 1491-1497.
2. Peer M, Lyon R, Arzy S (2014) Orientation and disorientation: Lessons from patients with epilepsy. Epilepsy Behav 41C: 149-157.
3. Buckner R L, Snyder A Z, Shannon B J, LaRossa G, Sachs R, et al. (2005) Molecular, structural, and functional characterization of Alzheimer's disease: evidence for a relationship between default activity, amyloid, and memory. J Neurosci 25: 7709-7717.
4. Buckner R L, Sepulcre J, Talukdar T, Krienen F M, Liu H, et al. (2009) Cortical hubs revealed by intrinsic functional connectivity: mapping, assessment of stability, and relation to Alzheimer's disease. J Neurosci 29: 1860-1873.
5. Seeley W W, Crawford R K, Zhou J, Miller B L, Greicius M D (2009) Neurodegenerative Diseases Target Large-Scale Human Brain Networks. Neuron 62: 42-52.
6. Arzy S, Molnar-Szakacs I, Blanke O (2008) Self in time: imagined self-location influences neural activity related to mental time travel. J Neurosci 28: 6502-6507.
7. Betti V, Della Penna S, de Pasquale F, Mantini D, Marzetti L, et al. (2013) Natural scenes viewing alters the dynamics of functional connectivity in the human brain. Neuron 79: 782-797.
8. Arzy S, Molnar-Szakacs I, Blanke O (2008) Self in time: imagined self-location influences neural activity related to mental time travel. J Neurosci 28: 6502-6507.
9. Buckner R L, Carroll D C (2007) Self-projection and the brain. Trends Cogn Sci 11: 49-57. 10.
10. Buckner R L, Andrews-Hanna J R, Schacter D L (2008) The brain's default network: anatomy, function, and relevance to disease. Ann N Y Acad Sci 1124: 1-38.
11. Gusnard D a, Akbudak E, Shulman G L, Raichle M E (2001) Medial prefrontal cortex and self-referential mental activity: relation to a default mode of brain function. Proc Natl Acad Sci USA 98: 4259-4264.
12. Kahn I, Andrews-Hanna J R, Vincent J L, Snyder A Z, Buckner R L (2008) Distinct cortical anatomy linked to subregions of the medial temporal lobe revealed by intrinsic functional connectivity. J Neurophysiol 100: 129-139.
13. Peer M. Nitzan M, Goldberg I, Gomori M, Arzy S (2014) Reversible functional disintegration of the episodic memory network during transient global amnesia. Ann Neurol 75: 634-43.
14. Arzy, S., Molnar-Szakacs, I., Blanke, O. (2008). J Neurosci. 28(25):6502-7.
15. Brunet, D., Murray, M. M., Michel, C. M. (2011). Comput Intell Neurosci. 2011:813870.

1. A method of neuropsychological analysis, the method comprising:
presenting to a subject, by a user interface, a subject-specific cognitive task having at least one task portion selected from the group consisting of a time-domain task portion, a space-domain task portion, and a person-domain task portion;
receiving responses entered by the subject using said user interface for each of said task portions;
representing said responses as a set of parameters; and
classifying said subject into one of a plurality of cognitive function classification groups, based on said set of parameters.

2. The method according to claim 1, wherein said subject-specific cognitive task comprises at least two of said time-domain, space-domain and person-domain task portions.

3. The method according to claim 1, wherein said subject-specific cognitive task comprises each of said time-domain, space-domain and person-domain task portions.

4. The method according to claim 1, further comprising constructing said subject-specific cognitive task.

5. (canceled)

6. The method according to claim 4, wherein said constructing said subject-specific cognitive task is executed automatically.

7. The method according to claim 6, further comprising receiving from a mobile device of the subject sensor data, wherein said subject-specific cognitive task is constructed based on said sensor data.

8. The method according to claim 6, further comprising accessing a social network account associated with said subject, and extracting social interaction data from said account, wherein said subject-specific cognitive task is constructed based on said social interaction data.

9. The method according to claim 6, further comprising receiving from a mobile device of the subject stored social interaction media, wherein said subject-specific cognitive task is constructed based on said stored social interaction media.

10. (canceled)

11. The method according to claim 1, further comprising receiving from a mobile device of the subject sensor data, wherein said classification is based also on said sensor data.

12-15. (canceled)

16. The method according to claim 1, further comprising receiving from a neurophysiological data acquisition system neurophysiological data pertaining to a brain of said subject, wherein said classification is based also on said neurophysiological data.

17. The method according to claim 1, further comprising accessing a library of reference data comprising at least parameters describing responses of previously classified subjects, and processing and analyzing said set of parameters using at least a portion of said reference parameters, wherein said classification is based also on said analysis.

18-20. (canceled)

21. The method according to claim 1, further comprising altering said cognitive task based on said responses, presenting said altered cognitive task to said subject, and receiving responses entered by the subject using said user interface for said altered cognitive task, wherein said classification is based on a comparison between responses entered before said alteration.

22. The method according to claim 1, further comprising presenting to said subject by said user interface, a feedback pertaining to at least one of said responses.

23. The method according to claim 22, further comprising re-presenting said cognitive task to said subject following said feedback, and receiving responses entered by the subject using said user interface for said re-presented cognitive task, wherein said classification is based on a comparison between responses entered before said feedback and responses entered after said feedback.

24. (canceled)

25. The method according to claim 1, further comprising presenting to a subject, by a user interface, at least one additional cognitive task, and receiving a response entered by the subject for each of said at least one additional task using said user interface for said at least one additional cognitive task, wherein said classifying is based also on said response to said at least one additional cognitive task.

26. (canceled)

27. The method according to claim 1, further comprising treating said subject for said classified cognitive function.

28. (canceled)

29. A server system for neuropsychological analysis, the server system comprising:
a transceiver arranged to receive and transmit information on a communication network; and
a processor arranged to communicate with the transceiver, and perform code instructions, comprising:
code instructions for transmitting to a client computer, a subject-specific cognitive task to be presenting to a subject by a user interface, said cognitive task having a time-domain task portion, a space-domain task portion, and a person-domain task portion;
code instructions for receiving from said client computer responses for each of said task portions;
code instructions for representing said responses as a set of parameters; and
code instructions for classifying said subject into one of a plurality of cognitive function classification groups, based on said set of parameters.

30. The system according to claim 29, wherein said processor is arranged to perform code instructions for:
constructing a subject-specific cognitive task having at least one task portion selected from the group consisting of a time-domain task portion, a space-domain task portion, and a person-domain task portion;
presenting said subject-specific cognitive task to a subject by a user interface;
receiving responses entered by the subject using said user interface for each of said task portions;
representing said responses as a set of parameters; and
classifying said subject into one of a plurality of cognitive function classification groups, based on said set of parameters.

31. The method according to claim 1, wherein said plurality of cognitive function classification groups comprises Mild Cognitive Impairment (MCI), Alzheimer's disease (AD), and age related cognitive decline.

32. The method according to claim 1, wherein said classifying comprises applying a domain-specific weight to each of said parameters.

33-34. (canceled)

35. The method according to claim 1, wherein said set of parameters comprises, for at least one of said task portions, a success rate and a response time.

36. The method according to claim 1, wherein at least one of said task portions comprises a first stimulus, a second stimulus and an instruction to rate a level of relationship between said subject and each of said stimuli.

37. The method according to claim 36, wherein at least two said of said task portions comprise different stimuli but similar instruction.

38. The method or system according to claim 1, wherein at least one of said task portions comprises a single assignment.

39. The method or system according to claim 1, wherein at least one of said task portions comprises a plurality of assignments.
